(19) United States
(12) Patent Application Publication
(10) Pub. No.: US 2015/0126627 A1
(43) Pub. Date: May 7, 2015

(54) INTEGRATION OF SYNGAS GENERATION TECHNOLOGY WITH FISCHER-TROPSCH PRODUCTION VIA CATALYTIC GAS CONVERSION

(71) Applicant: RES USA, LLC, Commerce City, CO (US)

(72) Inventors: George Apanel, Denver, CO (US); Weibin Jiang, Aurora, CO (US); Sergio Mohedas, Houston, TX (US); Harold A. Wright, Longmont, CO (US)

(21) Appl. No.: 14/414,535

(22) PCT Filed: Jul. 15, 2013
(86) PCT No.: PCT/US2013/050484

§ 371 Date: Jan. 13, 2015

Related U.S. Application Data
(60) Provisional application No. 61/672,023, filed on Jul. 16, 2012.

(30) Foreign Application Priority Data
Jul. 15, 2013 (WO) 2014014818

(51) Int. Cl. C07C 1/04; C01B 3/02; C10J 3/48

(52) U.S. Cl. CPC C07C 1/049; C10J 3/48; C07C 1/0485; C01B 3/02; C10J 2200/06; C01B 2203/0238; C01B 2203/062; C01B 2203/0805; C01B 2203/1058; C01B 2203/1235
USPC 518/702; 422/141

(57) ABSTRACT

A system for the production of synthetic fuel, the system including a catalytic dual fluidized bed (DFB) configured to produce, from a DFB feedgas, a DFB product containing synthesis gas; and a Fischer-Tropsch (FT) synthesis apparatus fluidly connected with the catalytic DFB, wherein the FT synthesis apparatus includes an FT synthesis reactor configured to produce, from an FT feedgas, an FT overhead and a liquid FT product containing FT wax, wherein the FT feedgas contains at least a portion of the DFB product; and a product separator downstream of and fluidly connected with the FT synthesis reactor, wherein the product separator is configured to separate, from the FT overhead, an FT tailgas and an LFTL product containing LFTL. A method of producing synthetic fuel is also provided.

### STATEMENT REGARDING FEDERALLY SPONSORED RESEARCH OR DEVELOPMENT

Not Applicable.

### TECHNICAL FIELD

The present invention generally relates to the production of synthetic hydrocarbons. More specifically, the present invention relates to the production of synthetic hydrocarbons via Fischer-Tropsch conversion of synthesis gas. Still more specifically, the present invention relates to the production of synthetic hydrocarbons via Fischer-Tropsch conversion of synthesis gas, at least a portion of which is produced via catalytic dual fluidized beds.

### BACKGROUND

In the context of its broadest general application, the term ‘gasification’ has generally been used to describe any process for the conversion of a solid, liquid, or vapor hydrocarbon, or more heterogeneous but predominantly carbonaceous compounds into synthesis gas (also referred to as syngas). The synthesis gas generally consists of a mixture of gases consisting predominantly of carbon monoxide and hydrogen. Low quality synthesis gas may be contaminated by methane, CO₂, and other impurities such as, but not limited to, aromatics and high molecular weight tars. These tars, a common byproduct of biomass gasification, can be particularly undesirable since, aside from representing a syngas yield loss, such tars can lead to serious fouling and plugging of downstream process equipment when cooled below their dewpoint, as may be required for downstream processing. When a gasification type process is applied as a secondary reaction stage for the conversion or removal of such impurities from a low quality synthesis gas, the term ‘conditioning’ is often used to describe such a secondary operation. Gasification or conditioning may therefore entail a wide variety of generic chemical reactions, which may include, without limitation, pyrolysis, cracking, partial oxidation, reforming, and/or shifting.

Depending on the particular carbonaceous feedstock, the impurities therein, and the specific reactions involved, such reactions may also entail the addition of steam and/or oxygen as a supplemental reactant(s) to help promote the desired reaction. Such processes may be conducted with or without the use of a catalytic or inert medium for the purpose of promoting efficient heat and mass transfer within the gasification reactor. Catalytic media may be particulate or monolithic in nature. A common drawback of some conventional gasification and proposed conditioning processes is byproduct soot formation, which can be even more problematic than tars, if sufficiently ‘sticky’ to foul downstream process equipment at even elevated temperature levels.

Gasification with pure steam in a fluidized bed is a highly endothermic process. For this reason, gasification, for example biomass gasification, has been combined with fluidized bed combustion to provide heat enthalpy and also to remove char formed during gasification. Such char may be undesirable in the gasification product gas. Dual fluidized bed gasification has thus been proposed in the art. Dual fluidized bed gasification is desirable due to the ability to produce high caloric product gas free of nitrogen dilution even when air is used to generate, via in situ combustion, the heat required by the endothermic gasification reactions.

Conventional gasification processes may be generally classified as either ‘direct’ or ‘indirect’. In direct gasification processes, an oxidant is directly contacted with a carbonaceous feed. In indirect gasification processes, an oxidant is supplied to a separate combustion reactor and is kept separate from the carbonaceous feed in the gasification reactor by a physical barrier, which may also serve as a heat transfer medium. The medium transfers heat from the exothermic combustion reactor to the endothermic gasification reactor, as exemplified by dual fluid gasification employing a circulating solid heat transfer medium. Another type of indirect gasification technology uses stationary heat transfer tubes to separate and transfer heat between the endothermic gasification and exothermic combustion reaction sections.

Direct gasification, which is currently widely practiced, generally utilizes three basic configurations which may be either air blown or oxygen blown: entrained flow (e.g. Siemens), fluidized bed (e.g. Winkler), and moving bed (e.g. Lurgi dry bottom). When air blown, the nitrogen component of the air undesirably dilutes the product synthesis gas, rendering it unsuitable for direct use in various downstream applications. For this reason, many direct gasifiers are oxygen-blown, requiring a source of high purity oxygen, which tends to be expensive. For example, an air separation unit is often utilized to provide the oxygen for an oxygen-blown gasifier. Despite the use of high purity oxygen, direct gasification, especially via fluid bed and moving bed gasifiers, often provides low-quality synthesis gas which, while appropriate for many applications, may be undesirable for downstream processes demanding high-quality synthesis gas. While oxygen blown entrained flow gasifiers may produce a better quality synthesis gas than produced by fluid bed or moving bed configurations, entrained flow gasifiers generally require more stringent feedstock preparation, which may not be practical for certain types of carbonaceous feeds, such as biomass or petroleum coke. Even if free from impurities, the synthesis gas produced by entrained flow or other types of gasification technologies may not meet a desired composition for certain downstream chemical or fuel synthesis applications. Further conditioning of such synthesis gas may therefore be required.

Indirect gasification technologies, particularly those based on dual fluidized beds, are generally known to produce low-quality synthesis gas comprising undesirably large amounts of impurities such as hydrogen, methane, carbon dioxide, and high dew point tars. Such technologies also generally mandate consumption of high levels of steam and other additives, such as dolomite, to promote gasification and maximize levels of quality product synthesis gas. Typically, undesirable components such as methane, carbon dioxide, excess hydrogen, tars, and/or sulfur and sulfur-containing components must be removed from low-quality synthesis gas produced via gasification prior to the use of the synthesis gas in downstream processes requiring chemical-grade synthesis gas. This contaminant removal may be costly, inefficient and complicated. The presence of such contaminants may also represent a substantial loss of potential product synthesis gas and downstream product yield if such contaminants are not converted to the high quality syngas required for certain chemical and fuel production processes.

As noted hereinabove, synthesis gas from gasification (e.g. biomass gasification, coal gasification), reforming (e.g. reforming of natural gas), and partial oxidation, typically contains significant amounts of unconverted carbon (e.g. tar, methane, and carbon dioxide). To avoid undesirable downstream operational issues, syngas cleanup/conditioning processes, which may be quite costly, are often needed to remove contaminants, such as carbon dioxide, prior to downstream operations, such as Fischer-Tropsch (FT) synthesis. For example, tar removal, such as via OLGA wash unit, may be utilized downstream of syngas production to remove tars from the synthesis gas prior to syngas compression, in order to ensure compressor operation. Once undesired contaminants are removed, and a desired flow of a cleaned-up/conditioned synthesis gas having a suitable composition (e.g. level of undesirable components, molar ratio of hydrogen to carbon monoxide, etc.) is obtained, the cleaned-up/conditioned syngas may be introduced into downstream processes.

In the case in which the synthesis gas is to be utilized downstream for the production of synthetic fuels via FT conversion, an uncondensed FT tailgas is often obtained, for example in overhead recovery operations, during FT synthesis. The FT tailgas typically contains unreacted hydrogen and carbon monoxide, along with carbon dioxide, light hydrocarbons (e.g. methane), and other inerts (e.g. nitrogen). To improve the overall carbon monoxide conversion of the FT process, a portion of the FT tailgas is sometimes recycled back to the FT reactor (i.e. as a component of the FT feedgas). However, such FT tailgas recycle is associated with a number of potential drawbacks. For example, carbon dioxide and oxygenate removal may be required prior to recycle of the FT tailgas. Also, if the FT tailgas has a higher molar ratio of hydrogen to carbon monoxide than the optimal ratio for downstream FT synthesis, recycle of the FT tailgas may undesirably affect (e.g. may increase) the molar ratio of hydrogen to carbon monoxide in the overall FT feedgas. Furthermore, recycle of FT tailgas may cause an accumulation of light hydrocarbons and/or inerts (e.g. methane, ethane, nitrogen, etc.), thus undesirably diluting the FT feed syngas, and increasing the volume flow rate, without increasing the overall FT production rate.

Processes associated with a plethora of industries result in the production or isolation of low value fuel gas. For example, low and/or medium BTU fuel gas may be a byproduct of coal mining and/or utilization (e.g. coal bed or coal mine methane, coal oven gas), fermentation (e.g. landfill gas), FT synthesis gas (FT tailgas), methanol production (e.g. LP methanol purge gas), oil mining and/or refining (e.g. stranded gas from an oil well, refinery offgas), and gas separation in any of the aforementioned and also a variety of other industries (e.g. PSA tailgas). Such byproduct gas may have little value and may typically be vented. Treatment at expense may be required in order to meet environmental regulations. Recovery of value from such gas usually involves two strategies: hydrocarbon recovery therefrom and/or conversion to process gas (e.g. conversion to synthesis gas and/or hydrogen).

As an example hydrocarbon recovery strategy, methane in landfill gas can be extracted therefrom, for example, via vacuum swing adsorption (VSA), and the extracted methane from VSA can be furthered enriched, for example via a cryogenic process, to produce liquid natural gas (LNG).

As an example conversion to process gas strategy, high temperature steam methane reforming may be utilized to convert carbon dioxide and methane in landfill gas into synthesis gas via reaction with excess steam. However, such steam methane reforming is associated with a number of potential drawbacks. For example, substantial landfill gas pretreatment may be required to remove and/or convert to a desired component(s) one or more undesired component(s) thereof (e.g. sulfur, heavy hydrocarbons) prior to introduction into an SMR unit. Also, the excess steam required for SMR may substantially reduce the overall plant fuel and/or thermal efficiency. Furthermore, the molar ratio of hydrogen to carbon monoxide in the SMR product syngas may not be appropriate for downstream processes, such as FT synthesis. Furthermore, carbon dioxide conversion may be unacceptably low.

In an integrated coal to liquids (CTL), biomass to liquids (BTL), or gas to liquids (GTL) plant employing FT technology, synthesis gas finds an array of uses. For example, synthesis gas may be utilized to provide hydrogen for product upgrading (e.g. hydroprocessing), synthesis gas may be utilized for activation of FT catalyst, power production, and etc. It is desirable, however, to improve the hydrogen and/or carbon monoxide usage efficiency of such processes.

Accordingly, there remains a need in the art for enhanced systems and methods of producing synthetic hydrocarbons. Desirably, such systems provide higher yields of synthesis gas via conversion of carbonaceous material(s), enable production of synthesis gas and subsequently of synthetic fuels from low value fuel gas, provide increased overall yields of synthetic fuels via conversion of synthesis gas, reduce or eliminate the need for extensive downstream cleaning of synthesis gas prior to downstream FT synthesis, allow for production of synthesis gas in the absence of costly air separation unit(s), and/or reduce and/or eliminate potential byproduct soot formation relative to conventional systems and methods.

### SUMMARY

Herein disclosed is a system for the production of synthetic fuel, the system comprising: a catalytic dual fluidized bed (DFB) configured to produce, from a DFB feedgas, a DFB product comprising synthesis gas; and a Fischer-Tropsch (FT) synthesis apparatus fluidly connected with the catalytic DFB, wherein the FT synthesis apparatus comprises: an FT synthesis reactor configured to produce, from an FT feedgas, an FT overhead and a liquid FT product comprising FT wax, wherein the FT feedgas comprises at least a portion of the DFB product; and a product separator downstream of and fluidly connected with the FT synthesis reactor, wherein the product separator is configured to separate, from the FT overhead, an FT tailgas and an LFTL product comprising LFTL. In embodiments, the system further comprises a fluid connection between the product separator and the catalytic DFB, whereby at least a portion of the FT tailgas can be introduced into the catalytic DFB. In embodiments, the system further comprises one or more apparatus selected from the group consisting of: gasification apparatus configured to produce synthesis gas from a gasifier feed; compressors upstream of the FT synthesis reactor and configured to compress at least a portion of the FT feedgas; syngas conditioning apparatus selected from the group consisting of tar removal apparatus, CO₂ removal apparatus, sulfur removal apparatus, and combinations thereof, wherein the syngas conditioning apparatus is located upstream of and is fluidly connected with the FT synthesis reactor; heat recovery apparatus downstream of and fluidly connected with the catalytic DFB and configured to recover heat from the DFB product gas; heat recovery apparatus downstream of and fluidly connected with the FT synthesis reactor and configured to recover heat from the FT overhead; solid/gas separators upstream of the catalytic DFB and configured to remove solids from at least a portion of the DFB feedgas; solid/gas separators downstream of the catalytic DFB and configured to remove solids from at least a portion of the DFB product gas; and product upgrading apparatus downstream of and fluidly connected with the product separator, wherein the product upgrading apparatus is configured to upgrade at least a portion of the LFTL product, at least a portion of the liquid FT product, or at least a portion of both the LFTL product and the liquid FT product, thus providing one or more synthetic fuels. In embodiments, the system comprises at least one each of each of the apparatus listed previously. In embodiments, the syngas conditioning apparatus comprises no tar removal apparatus.

In embodiments, the system is configured for introduction of the at least a portion of the FT tailgas into the catalytic DFB as a fuel, as a feedgas, or both.

In embodiments, the catalytic DFB comprises: a fluid bed conditioner operable to produce the DFB product gas from the DFB feedgas, wherein the fluid bed conditioner comprises an outlet for a first catalytic heat transfer stream comprising a catalytic heat transfer material and having a first temperature, and an inlet for a second catalytic heat transfer stream comprising catalytic heat transfer material and having a second temperature that is greater than the first temperature; a fluid bed combustor operable to combust fuel and oxidant introduced thereto, wherein the fluid bed combustor comprises an inlet fluidly connected with the outlet for a first catalytic heat transfer stream of the conditioner, and an outlet fluidly connected with the inlet for a second catalytic heat transfer stream of the fluid bed conditioner; and a catalytic heat transfer material. In embodiments, the catalytic heat transfer material comprises a supported or unsupported metal catalyst. In embodiments, the catalytic heat transfer material comprises a supported or unsupported nickel catalyst. In embodiments, the catalytic heat transfer material comprises a supported catalyst, and the support is selected from the group consisting of alumina, olivine, silica, and combinations thereof.

In embodiments, the DFB feedgas comprises a low quality synthesis gas, wherein the low quality synthesis gas comprises a greater percentage of non-syngas components than the DFB product gas, and the system further comprises a gasifier operable to produce the low quality synthesis gas, wherein the gasifier is located upstream of the fluid bed conditioner and fluidly connected therewith, whereby at least a portion of the low quality synthesis gas may be introduced into the fluid bed conditioner as DFB feedgas. The gasifier may be one fluid bed of a dual fluidized bed gasification apparatus. The dual fluidized bed gasification apparatus may comprise: a fluid bed gasifier operable to produce low quality synthesis gas from carbonaceous material and optionally steam, and comprising an outlet for a first heat transfer stream comprising a heat transfer material and unconverted carbonaceous material and having a third temperature, and an inlet for a second heat transfer stream comprising heat transfer material and having a fourth temperature greater than the third temperature; a second fluid bed combustor operable to combust oxidant and fuel and produce a flue gas, wherein the second fluid bed combustor comprises a second fluid bed combustor inlet fluidly connected with the outlet for a first heat transfer material stream of the fluid bed gasifier, and a second fluid bed combustor outlet fluidly connected with the inlet for a second heat transfer stream of the fluid bed gasifier; and a heat transfer material.

In embodiments, the carbonaceous material is selected or derived from a material selected from the group consisting of biomass, municipal sludge, RDF, coal, petroleum coke, natural gas, E-FUEL, and combinations thereof.

In embodiments, the system further comprises a fluid connection between the fluid bed conditioner and the product separator, whereby at least a portion of the FT tailgas can be introduced into the fluid bed conditioner as at least one carbon-containing component of the DFB feedgas. The system may be configured such that the DFB feedgas comprises substantially no carbon-containing gas other than the FT tailgas. In embodiments, the FT tailgas comprises carbon dioxide and at least one component selected from methane, ethane, propane, and higher hydrocarbons (including, without limitation, C2+ oxygenates, olefins and others), and the catalytic DFB is operable to continuously dry reform the DFB feedgas to produce the DFB product comprising synthesis gas.

In embodiments, the system is configured for the introduction of additional synthesis gas, not produced in the catalytic DFB, into the FT synthesis reactor, whereby the additional synthesis gas and the at least a portion of the DFB product gas can be introduced into the FT synthesis reactor as FT feedgas. The additional synthesis gas may be produced via gasification, reforming, partial oxidation, or a combination thereof.

The disclosed system may further comprise one or more apparatus selected from the group consisting of: compressors upstream of the FT synthesis reactor and configured to compress at least a portion of the FT feedgas; heat recovery apparatus downstream of and fluidly connected with the FT synthesis reactor and configured to recover heat from the FT overhead; and product upgrading apparatus downstream of and fluidly connected with the product separator, wherein the product upgrading apparatus is configured to upgrade at least a portion of the LFTL product, at least a portion of the liquid FT product, or at least a portion of both the LFTL product and the liquid FT product, thus providing one or more synthetic fuels. In embodiments, the system comprises at least one of each of the apparatus listed immediately previous.

In embodiments, the disclosed system comprises one or more apparatus selected from the group consisting of: gasification apparatus configured to produce synthesis gas from a gasifier feed; compressors upstream of the FT synthesis reactor and configured to compress at least a portion of the FT feedgas; syngas conditioning apparatus selected from the group consisting of tar removal apparatus, CO₂ removal apparatus, sulfur removal apparatus, and combinations thereof, wherein the syngas conditioning apparatus is located upstream of and is fluidly connected with the FT synthesis reactor; heat recovery apparatus downstream of and fluidly connected with the catalytic DFB and configured to recover heat from the DFB product gas; heat recovery apparatus downstream of and fluidly connected with the FT synthesis reactor and configured to recover heat from the FT overhead; solid/gas separators upstream of the catalytic DFB and configured to remove solids from at least a portion of the DFB feedgas; solid/gas separators downstream of the catalytic DFB and configured to remove solids from at least a portion of the DFB product gas; and product upgrading apparatus downstream of and fluidly connected with the product separator, wherein the product upgrading apparatus is configured to upgrade at least a portion of the LFTL product, at least a portion of the liquid FT product, or at least a portion of both the LFTL product and the liquid FT product, thus providing one or more synthetic fuels. The system may comprise at least one each of each of the apparatus listed immediately previous, and the gasification apparatus may comprise an indirect biomass gasifier fluidly connected with the catalytic DFB. In embodiments, the syngas conditioning apparatus comprises no tar removal apparatus.

In embodiments, the system is configured for the introduction into the catalytic DFB of a DFB feedgas comprising one or more gas selected from the group consisting of low BTU fuel gases and medium BTU fuel gases, and/or the catalytic DFB is operable to continuously dry reform the DFB feedgas to produce the DFB product comprising synthesis gas. The DFB feedgas may consist essentially of no other carbon-containing gas other than one or more gas selected from the group consisting of low BTU fuel gases and medium BTU fuel gases, and FT tailgas. The DFB feedgas may consist essentially of no other carbon-containing gas other than one or more gas selected from the group consisting of low BTU fuel gases and medium BTU fuel gases.

Also disclosed herein is a method of producing synthetic fuel, the method comprising: producing a dual fluidized bed (DFB) product from a DFB feedgas, via a catalytic DFB, wherein the DFB product comprises synthesis gas; introducing an FT feedgas comprising at least a portion of the DFB product into an FT synthesis reactor; extracting a gaseous FT overhead and a liquid FT product comprising FT wax from the FT synthesis reactor; separating, from the FT overhead, an FT tailgas and an LFTL product comprising LFTL; and upgrading at least a portion of the LFTL product, at least a portion of the liquid FT product, or at least a portion of both the LFTL product and the liquid FT product, thus providing one or more synthetic fuels. The method may further comprise introducing at least a portion of the FT tailgas into the catalytic DFB. The at least a portion of the FT tailgas may be introduced into the catalytic DFB as a fuel, as at least a component of the DFB feedgas, or both.

In embodiments of the disclosed method, producing a DFB product from a DFB feedgas further comprises introducing the DFB feedgas into a fluid bed conditioner, wherein the fluid bed conditioner is configured to convert at least a portion of said DFB feedgas into synthesis gas; extracting a first catalytic heat transfer stream comprising a catalytic heat transfer material and having a first temperature from the fluid bed conditioner and introducing at least a portion of the first catalytic heat transfer stream and a flue gas into a fluid bed combustor, wherein the fluid bed combustor is configured to regenerate the catalyst; extracting a second catalytic heat transfer stream comprising catalytic heat transfer material and having a second temperature from the fluid bed combustor and introducing at least a portion of the second catalytic heat transfer stream into the fluid bed conditioner; and extracting the DFB product from the fluid bed conditioner The catalytic heat transfer material may comprise a supported or unsupported metal catalyst. The catalytic heat transfer material may comprise a supported or unsupported nickel catalyst. The catalytic heat transfer material may comprise a supported catalyst, and the support may be selected from the group consisting of alumina, olivine, silica, and combinations thereof.

The method may further comprise introducing at least a portion of the FT tailgas into the fluid bed conditioner as at least a component of the DFB feedgas. The FT feedgas may further comprise additional synthesis gas not produced in the catalytic DFB. The additional synthesis gas may be produced via gasification, reforming, partial oxidation, or a combination thereof. The DFB feedgas may comprise substantially no carbon-containing gas other than the FT tailgas. The FT tailgas may comprise carbon dioxide and at least one component selected from the group consisting of methane, ethane, propane, and higher hydrocarbons (including, without limitation, C2+ oxygenates, olefins and others), and the catalytic DFB may be operable to continuously dry reform the DFB feedgas to produce the DFB product comprising synthesis gas.

The method may further comprise producing low quality synthesis gas by gasifying a carbonaceous material, and the DFB feedgas may comprise at least a portion of the low quality synthesis gas. The carbonaceous material may be derived from or selected from the group consisting of biomass, municipal sludge, RDF, coal, petroleum coke, natural gas, E-FUEL and combinations thereof.

In embodiments, gasifying a carbonaceous material comprises: introducing the carbonaceous material into a fluid bed gasifier of a dual fluidized bed gasification apparatus, wherein the carbonaceous material is gasified under gasification conditions; extracting a first heat transfer stream comprising heat transfer media and any unconverted carbonaceous material from the fluid bed gasifier and introducing at least a portion of the first heat transfer stream into a second fluid bed combustor, wherein the first heat transfer stream has a third temperature; introducing oxidant and fuel into the second fluid bed combustor whereby unconverted carbonaceous material in the first heat transfer stream is combusted and the temperature of the heat transfer media is raised; extracting a second heat transfer stream comprising heat transfer media and having a fourth temperature that is greater than the third temperature from the second fluid bed combustor and introducing at least a portion of the second heat transfer stream into the fluid bed gasifier; and extracting low-quality synthesis gas from the fluid bed gasifier.

In embodiments, the disclosed method further comprises operating the fluid bed combustor at from about 1 to 1.1 times stoichiometric air.

In embodiments, the method further comprises removing at least one component selected from the group consisting of tar, carbon dioxide, and sulfur from the at least a portion of the DFB product prior to introduction thereof into the FT synthesis reactor. In embodiments, the method comprises no additional tar removal from the at least a portion of the DFB product prior to introduction thereof into the FT synthesis reactor.

In embodiments of the disclosed method, the DFB feedgas comprises one or more gas selected from the group consisting of low BTU fuel gases and medium BTU fuel gases, and/or the catalytic DFB is operable to continuously dry reform the DFB feedgas to produce the DFB product comprising synthesis gas. In embodiments, the DFB feedgas comprises no carbon-containing gas other than one or more carbon-containing gas selected from the group consisting of low BTU fuel gases, medium BTU fuel gases, FT tailgas, and combinations thereof. In embodiments, the DFB feedgas comprises no carbon-containing gas other than one or more carbon-containing gas selected from the group consisting of low BTU fuel gases and medium BTU fuel gases.

These and other embodiments and potential advantages will be apparent in the following detailed description and drawings.

### BRIEF DESCRIPTION OF THE DRAWINGS

For a detailed description of the preferred embodiments of the invention, reference will now be made to the accompanying drawings in which:

FIG. 1 is a schematic of a system for the production of synthetic hydrocarbons, according to an embodiment of this disclosure;

FIG. 2 is a schematic of a system for the production of synthetic hydrocarbons, according to another embodiment of this disclosure;

FIG. 3 is a schematic of a system for the production of synthetic hydrocarbons, according to another embodiment of this disclosure;

FIG. 4 is a schematic of a system for the production of synthetic hydrocarbons, according to another embodiment of this disclosure; and

FIG. 5 is a schematic of a dual fluidized bed reactor system, according to an embodiment of this disclosure.

In the figures, like numbers are utilized to refer to like components.

### NOTATION AND NOMENCLATURE

As used herein, the term ‘Light Fischer-Tropsch Liquids’ or ‘LFTL’ is used to refer to mixtures enriched with C5-C30 alkanes, which may also contain olefins and oxygenated compounds, such as alcohols or acids, which may be present, for example, in the FT tailgas.

The phrase ‘higher hydrocarbons’ is generally used herein to refer to all hydrocarbon-bearing compounds other than methane, including, without limitation, olefins, oxygenates, mercaptans, thiophenes, and heteroatom hydrocarbon compounds.

As used herein, a ‘low BTU’ fuel gas is a fuel gas having a heating value between 90 and 300 BTU per cubic foot.

As used herein, a ‘medium BTU’ fuel gas is a fuel gas having a heating value between 300 and 600 BTU per cubic foot.

Unless otherwise stated, concentrations herein are expressed on a volume basis. That is ppm means ppmv, unless otherwise indicated.

‘Syngas yield’ as used herein is defined as the relative quantity of syngas produced with a minimum molar ratio of H₂ to CO required for a particular product application, for a particular quantity of gasifier or conditioner feedstock. For FT liquids production via slurry phase iron catalysis, a claimed increase in syngas yield of 100%, for example, would mean doubling the quantity of CO produced assuming sufficient H₂ is also produced for the desired equimolar ratio.

As used herein, the ‘yield’ of FT liquids from a carbonaceous feed material is defined as the ratio of desired product to material feed, typically stated as percent or fraction of material feed and assuming 100% conversion of the carbonaceous feed material. The product is often also described in volumetric units, whereas the feed can be expressed in mass units under certain assumed standard conditions. For example, in an FT liquids plant utilizing biomass as the carbonaceous feed material, the yield of product liquids may be expressed in terms of barrels of liquid product per ton of biomass feed on a moisture free basis.

Use herein of the terms ‘hot’ and ‘cold’ when used in reference to circulating fluid streams is meant to refer to relative, rather than absolute, temperatures.

Use herein of the terms ‘low’ and ‘high’ when used in reference to the quality of synthesis gas is meant to refer to relative, rather than absolute quality of the synthesis gas. That is, ‘low’ quality synthesis gas contains a higher content of contaminants (components other than hydrogen and carbon monoxide) than does ‘high’ quality synthesis gas.

### DETAILED DESCRIPTION

Overview.

Herein disclosed are a system and method for the production of synthetic hydrocarbons. The system and method incorporate a catalytic dual fluidized bed loop to provide and/or produce synthesis gas, from which synthetic hydrocarbons and/or other desired products may be produced. In embodiments, the system and method integrate synthesis gas generation technology with FT technology via catalytic gas conversion technology. In embodiments, the yield of FT liquids from a carbonaceous feed material (yield defined here as barrels per dry ton of carbonaceous feed material) may be increased by utilization of the disclosed system and method. In embodiments, the yield of FT liquids is increased by at least 10, 20, 30, 40, 50, 60, 70, 80, 90, or 100% relative to conventional gasification in the absence of catalytic dual fluid bed reforming.

In embodiments of the disclosed system and method, Fischer-Tropsch tailgas is put to use as a feed and/or fuel to a catalytic dual fluidized bed loop. Such process integration may enable enhanced overall recovery of hydrogen and carbon monoxide in the FT tailgas. In this manner, the overall yield of an integrated process (e.g. a biomass refinery process) may be improved. Passage of the FT tailgas through the catalytic gas conversion apparatus prior to recycle to FT synthesis apparatus converts non synthesis gas components therein to synthesis gas components, via dry reforming, and enables reintroduction of the synthesis gas components of the FT tailgas into the FT synthesis apparatus. Passage of FT tailgas through the catalytic gas conversion apparatus (e.g. catalytic dual fluidized bed) may eliminate or reduce the size/extent of expensive unit operations, such as, for example, carbon dioxide and/or tar removal from FT tailgas prior to recycle to FT synthesis. Additionally, conversion of carbon dioxide in the FT tailgas into synthesis gas via such dry reforming may reduce the level of undesirable carbon dioxide emissions.

In embodiments of the disclosed system and method, fuel gas (e.g. low and/or medium BTU value fuel gas) containing carbon dioxide and one or more hydrocarbon is put to use as a feed and/or fuel to a catalytic dual fluidized bed. Such utilization of generally low value gas may enable the production of additional synthesis gas, which can be further converted to FT hydrocarbons via FT synthesis, and subsequently upgraded to synthetic fuel(s). As mentioned hereinabove with respect to FT tailgas, such process integration may enable a reduction in carbon dioxide emissions, for which ever more stringent regulatory limits are expected.

System.

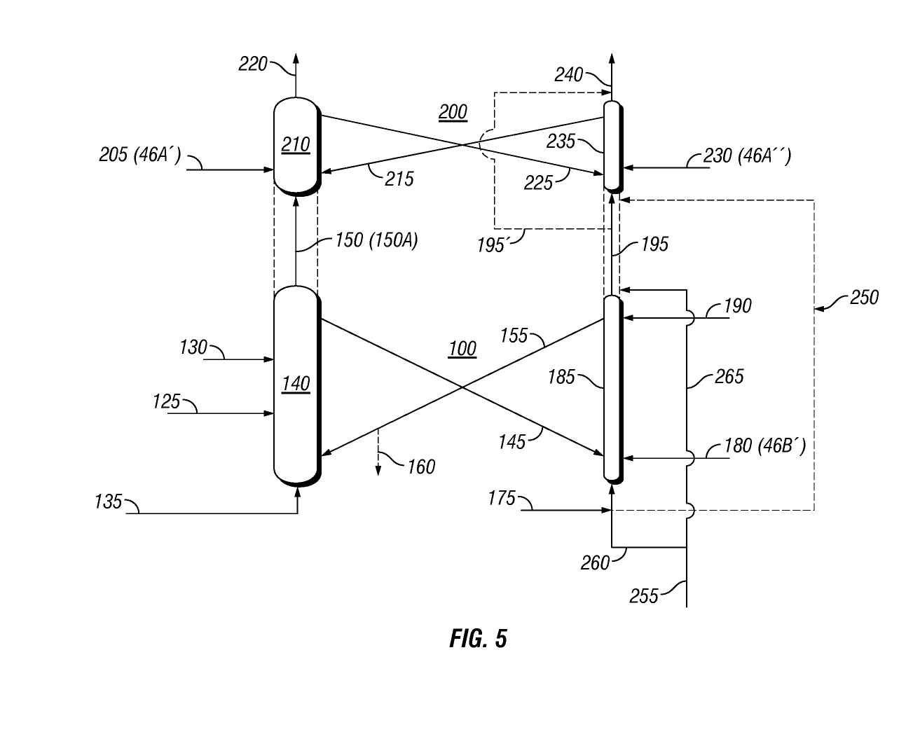

Herein disclosed is a system for the production of synthetic hydrocarbons. The system comprises a catalytic dual fluidized bed (DFB) and a Fischer-Tropsch (FT) synthesis apparatus fluidly connected therewith, such that at least a portion of the synthesis gas conditioned and/or produced in the catalytic DFB is introduceable into the FT apparatus, as at least a portion of a feedgas thereto. The catalytic DFB is configured to provide a DFB product comprising synthesis gas from a DFB feedgas, by converting non-synthesis gas components of the DFB feedgas (e.g. tar, methane, carbon dioxide) into synthesis gas. Suitable catalytic dual fluidized beds are described in U.S. patent application Ser. No. 12/691,297, filed Jan. 21, 2010, and now U.S. Pat. No. 8,241,523, and further described hereinbelow with reference to FIG. 5. U.S. Pat. No. 8,241,523 is hereby incorporated herein for all purposes not contrary to this disclosure. Any suitable FT synthesis apparatus known to those of skill in the art may be employed. In embodiments, the FT synthesis apparatus comprises at least one FT synthesis reactor configured to convert synthesis gas into FT hydrocarbons, thus producing a gaseous FT overhead comprising vaporized light Fischer-Tropsch liquids (LFTL), and a liquid FT product comprising molten FT wax; and a product separator configured to separate, from the FT overhead, an FT tailgas and an LFTL product comprising LFTL.

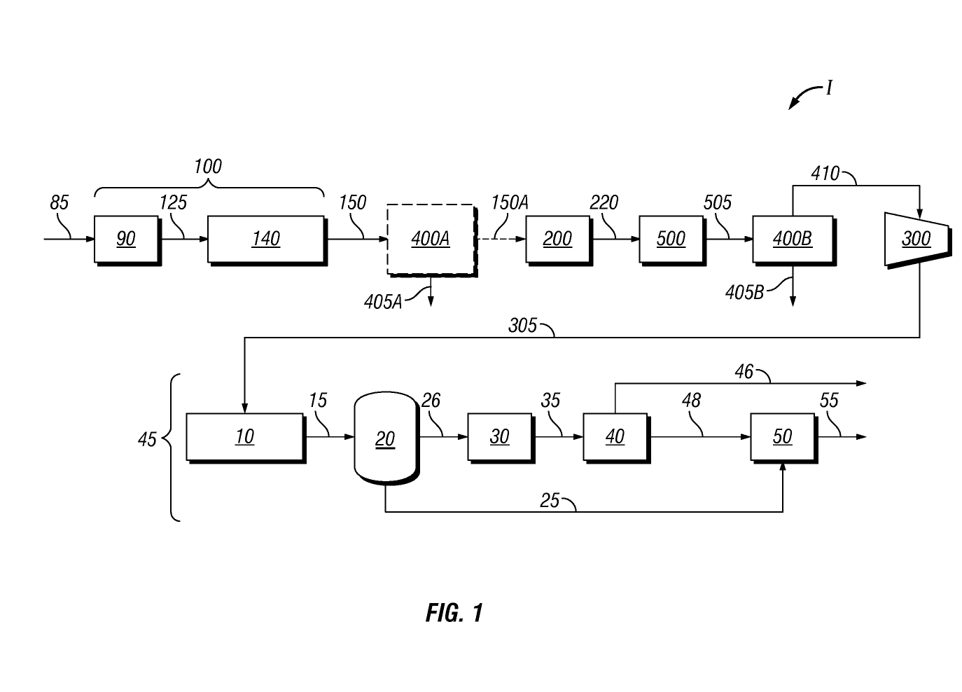

As seen in FIGS. 1, 2, 3 and 4, which depict systems I, II, III, and IV, respectively, according to embodiments of this disclosure, the herein disclosed system comprises a catalytic DFB 200 integrated with an FT synthesis apparatus 45. As noted hereinabove, FT synthesis apparatus 45 comprises at least one FT reactor 20, and at least one product separator 40. The FT synthesis apparatus may further comprise synthesis gas conditioning apparatus 10 positioned upstream of and fluidly connected with FT synthesis apparatus 20, overhead heat recovery apparatus 30 downstream of and fluidly connected with FT synthesis apparatus 20 and configured to recover heat from an FT overhead, and/or product upgrader 50 downstream of and fluidly connected with product separator 40. A system of this disclosure may further comprise one or more components selected from the group consisting of gasification apparatus 100 upstream of catalytic DFB 200 and configured to produce synthesis gas from a gasifier feed; compressors 300 upstream of FT synthesis apparatus 20 and configured to compress at least a portion of the FT feedgas; heat recovery apparatus 500 downstream of and fluidly connected with catalytic DFB 200 and configured to recover heat from a DFB product gas; solid/gas separators 400A upstream of catalytic DFB 200 and configured to remove solids from at least a portion of a DFB feedgas; solid/gas separators 400B downstream of catalytic DFB 200 and configured to remove solids from at least a portion of a DFB product gas; and recycle lines 46A fluidly connecting catalytic DFB 200 with FT synthesis apparatus 45 (e.g. with product separator 40). In embodiments, gasification apparatus 100 comprises a gasifier 140, and a carbonaceous feed handling apparatus 90 located upstream of gasifier 140 and configured to prepare and/or introduce an appropriate carbonaceous feed material thereto. Each of these components will be described in more detail hereinbelow.

Catalytic Gas Conversion Apparatus.

As noted hereinabove, a system of this disclosure comprises a catalytic gas conversion apparatus 200. Any suitable catalytic gas conversion apparatus known in the art may be utilized. Desirably, catalytic gas conversion apparatus is a catalytic DFB as described in U.S. patent application Ser. No. 12/691,297, filed Jan. 21, 2010, and now U.S. Pat. No. 8,241,523. Catalytic gas conversion apparatus 200 may be referred to herein as catalytic DFB 200, but it should be understood that other suitable gas conversion apparatus known in the art or invented in the future may be employed. Such a catalytic DFB will now be described in detail with reference to FIG. 5.

The catalytic DFB is configured to produce synthesis gas from a non-synthesis gas feed and/or from non-synthesis gas components of a DFB feedgas. The catalytic DFB may be referred to herein as a conditioner and may be utilized to condition a low quality synthesis gas (also referred to herein as ‘syngas’). For example, such a catalytic DFB may be operable to convert a low quality synthesis gas containing excessive levels of methane, higher hydrocarbons, tars, and/or carbon dioxide (e.g. ‘natural gas’ comprising synthesis gas) into a higher quality synthesis gas suitable for chemical feedstock applications, such as Fischer-Tropsch (FT) processes. Alternatively, or additionally, the catalytic DFB may be operable to produce synthesis gas from non-synthesis gas components of a DFB feedgas. For example, the catalytic DFB may be operable to dry reform a DFB feedgas comprising carbon dioxide and one or more hydrocarbons, such as, but not limited to methane, ethane, and/or propane, thus producing synthesis gas therefrom. Thus, the DFB feedgas may or may not contain synthesis gas.

With reference now to FIG. 5, in embodiments, the catalytic DFB comprises a dual fluid bed (DFB) conditioner/reformer loop in which an attrition resistant catalytic heat transfer medium is circulated between an endothermic reforming/conditioning/gasification reactor and an exothermic air blown combustion reactor.

The catalytic dual fluidized bed 200 depicted in FIG. 5 and described in detail hereinbelow, may sometimes be referred to herein as a ‘reforming loop’, or a ‘DFB conditioning loop’, and, as noted hereinabove, conditioner 210 may sometimes be referred to herein as a gasifier 210, conditioner 210, reformer 210, or fuel reactor 210. Combustion reactor 235 may also be referred to herein as combustor 235, regenerator 235, or air reactor 235. It is to be understood that, although referred to at times herein as a reforming loop or a reformer, in embodiments the reforming loop and/or reformer promote reactions other than reforming, such as, but not limited to, pyrolysis, cracking, partial oxidation and/or shifting. In embodiments, the conditioning reactor is a steam reforming reactor.

Gas conversion apparatus or DFB 200 may be operable with a heat transfer medium. The heat transfer medium may comprise a nickel-rich catalytic heat transfer medium, such as nickel olivine, or a more attrition resistant nickel alumina catalyst, or any other fluidizable, attrition resistant, supported or unsupported (i.e. heterogeneous or homogeneous) catalyst with suitable hydrocarbon and CO₂ reforming and CO shift activity. Suitable nickel alumina catalyst is disclosed, for example, in international patent application number PCT/US2005/036588, which is hereby incorporated herein in its entirety for all purposes not contrary to this disclosure. In embodiments, the hot catalyst endothermically reforms components of the DFB feedgas, optionally in the presence of steam, while the combustor exothermally regenerates the circulating catalyst by burning off any residual coke. Supplemental fuel may be utilized in the combustor, if necessary. In this manner, nitrogen in the combustion air proceeds into the combustor flue gas and does not dilute the DFB product synthesis gas, and the bed material of the conditioner is not diluted with ash. The supplemental fuel to the combustor in the DFB reformer loop may be any low sulfur gas which supports combustion.

The disclosed DFB reactor concept resembles conventional petroleum refinery fluid catalytic cracking (FCC) technology in some respects and reduces and/or eliminates drawbacks typical of conventional reforming technologies when applied as disclosed to conditioning/reforming of low quality synthesis gas, and/or non-syngas DFB feedgas.

System.

The catalytic dual fluid bed reforming loop 200 for the production/conditioning of synthesis gas according to this disclosure comprises a conditioner/reformer 210 coupled with a combustion reactor 235. Conditioner 210 is any suitable fluidized bed reformer known in the art. Conditioner/reformer 210 is configured to react methane, higher hydrocarbons, tars, and/or CO₂ in the DFB feedgas (e.g. crude synthesis gas) to produce hydrogen and carbon monoxide. In this manner, the DFB product of reformer 210 comprises synthesis gas produced therein and optionally also synthesis gas introduced thereto (i.e. passing therethrough). The DFB product syngas may have a desired molar ratio of H₂:CO, as discussed further hereinbelow.

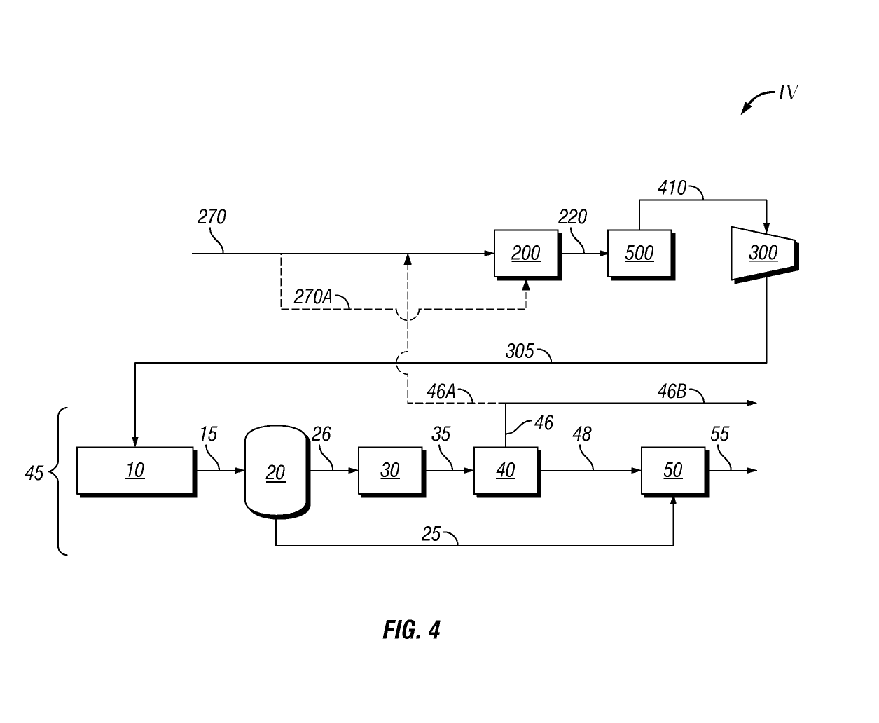

DFB feedgas or ‘conditioner inlet’ line 150 (or 150A) is configured to introduce a gas to be conditioned (i.e. a low-quality synthesis gas) and/or a gas to be converted into synthesis gas (e.g. low BTU fuel gas, any other gas containing reformable components) into conditioner 210. DFB feedgas in line 150 may be obtained by any means known in the art. In embodiments, the DFB feedgas in line 150 comprises low-quality synthesis gas. In embodiments, the DFB feedgas comprises low and/or medium BTU fuel gas, as discussed in more detail with reference to the embodiment of FIG. 4. The DFB feedgas may comprise significant amounts of methane, tar, and/or compounds comprising two or more carbons. In embodiments, methane levels in the DFB feedgas may in the range of, or even higher than, about 10 to about 15 volume percent, C₂ and higher hydrocarbon levels may be in the range of, or even higher than, about 5 to about 10 volume percent, CO₂ levels may be in the range of, or even higher than, about 5 to about 20 volume percent, and/or tar levels may be in the range of, or even higher than, about 1,000 to about 50,000 mg/Nm³. In embodiments, substantial catalyst activity is maintained at tar concentrations above those conventionally reported for DFB gasifiers (e.g. up to at least about 125,000 mg/Nm³ (over three times that normally reported for a SilvaGas DFB gasifier, for example), with destruction of the tars down to measurable levels of less than or equal to about 1 mg/Nm³ in the high quality DFB product synthesis gas exiting the conditioner. Such tar reduction is due to reforming of the tars into syngas via the DFB.

In applications, additional material to be reformed is introduced into conditioner/reformer 210 along with DFB feedgas in line 150. For example, in applications for which the synthesis gas is to be used for the production of liquid hydrocarbons via Fischer-Tropsch, FT tailgas comprising unconverted synthesis gas and other gases may be introduced into reformer 210 along with the DFB feedgas in line 150. Desirably, feed materials (e.g. crude low-quality synthesis gas in line 150 and/or recycle tailgas which may be fed into conditioner 210 or line 150 via an FT tailgas recycle line 205(46A′)) to conditioner/reformer 210 comprise little or no carbonaceous solids or residual ash, as such materials may, depending on the catalyst, hinder catalyst performance. Depending on the selected catalyst, maximizing concentration of the catalyst in the catalyst bed of conditioner/reformer 210 may enable increased/maintained catalyst performance. Within conditioner 210, steam and carbon dioxide and lighter hydrocarbons such as natural gas (methane) may react (e.g. be reformed) to produce synthesis gas.

In the embodiment of FIG. 5, bed material from conditioner 210 is circulated around dual fluid bed loop 200 via ‘cold’ bed material outlet line 225 which introduces ‘cold’ bed material from conditioner 210 into combustion reactor 235, while ‘hot’ bed material is returned to conditioner 210 via ‘hot’ bed material return line 215. As mentioned hereinabove, the terms ‘cold’ and ‘hot’ with reference to bed material indicate the temperature of one relative to the other. Although referred to as ‘cold’, the material therein may be at significant temperatures not normally considered cold, as further discussed hereinbelow.

Suitable circulation rates may be determined in part as a function of the differential temperature of the ‘hot’ and ‘cold’ streams. Operation of the DFB(s) may provide a differential temperature in the range of from about 25° F. (16° C.) to about 300° F. (149° C.), and may be about 150° F. (83° C.) in certain applications. Generally, the greater the temperature differential, the less the material that needs to be circulated between the reactors to maintain the desired endothermic gasifier/conditioner temperature(s).

Within combustion reactor or combustor/regenerator 235, flue gas comprising excess air introduced into combustion reactor 235 via flue gas inlet line 195 is combusted, optionally with additional fuel introduced into combustion reactor 235 via, for example, fuel inlet line 230 (46A″). In embodiments, as indicated in FIG. 5, a portion or the entire quantity of the flue gas stream 195 may bypass combustor/regenerator 235, as indicated by dashed line 195′, while a portion or the entire quantity of oxidant is supplied directly to combustor/regenerator 235, as indicated by dashed line 250. In embodiments, fuel introduced via line 230 (46A″) comprises tailgas, e.g. FT tailgas, from line 46A, for example, as discussed further hereinbelow. Flue gas introduced into combustor 235 via line 195 may contain some sulfur dioxide, for example from about 0 to about 50 ppmv, from about 5 to about 40 ppmv, or from about 10 to about 30 ppmv SO₂. Significant amounts of ash are not expected to be present in catalytic DFB loop 200, providing a potential advantage thereof. However, any coke and ash remaining in/on the ‘cold’ bed material is subjected to the combustion conditions within combustor 235 (and inorganic constituents of the ash are oxidized or reduced), heating the bed material therein. Heated/regenerated bed material (i.e. ‘hot’ bed material reduced in ash, coke, and/or other contaminants) is returned to conditioner/reformer 210 via ‘hot’ bed material return line 215. As discussed further herein, fluidized bed combustor 235 may be operable at a temperature in the range of from about 880° C. to about 925° C. or from about 910° C. to about 915° C., and flue gas in line 240 may thus exit combustor 235 at such temperature. This may be referred to herein as the ‘regeneration’ temperature.

As mentioned hereinabove, the bed material circulated throughout dual fluid bed loop 200 may comprise any suitable heat transfer medium comprising a catalyst capable of catalyzing reformation of materials, such as, but not limited to, natural gas and/or carbon dioxide. In embodiments, the bed material comprises an attrition resistant nickel olivine catalyst, such as that developed by the University of Strasbourg (France) and demonstrated for gasifying low sulfur biomass feeds. In applications, the bed material comprises a nickel alumina catalyst. As mentioned hereinabove, suitable catalyst is disclosed in international patent application number PCT/US2005/036588.

The DFB feedgas may comprise greater than about 20 volume percent, 25 volume percent, 30 volume percent, or greater impurities (e.g. tar, hydrogen sulfide, and/or other non-synthesis gas components). In embodiments, the catalyst and/or system is operable at gas sulfide concentrations of up to at least 10 ppm, at least 50 ppm, at least 100 ppm, or at least 200 ppm, without deactivation or substantial loss of catalyst (e.g. nickel catalyst) activity. In embodiments, the DFB feedgas in line 150 has a sulfur concentration of at least 10, 50, 100, 200, 300, 400, 500, 600, 700, 800, 900, or 1000 ppmv. In embodiments, the hydrogen sulfide concentration in the gas to be conditioned is up to 1000 ppmv, and the catalyst retains at least some activity (although activity will generally be reduced at higher sulfide concentrations). In embodiments, the feed to the conditioner of DFB conditioning loop 200 comprises substantial amounts of tar and substantially all of the tar is destructed/converted/reformed to synthesis gas within the DFB. In embodiments, the catalyst and/or system is operable at tar concentrations of at least 50,000 mg/Nm³, 60,000 mg/Nm³, or 70,000 mg/Nm³, without catalyst deactivation or substantial loss of catalyst activity. In embodiments, the DFB feedgas in line 150 contains at least 50,000 mg/Nm³, 60,000 mg/Nm³, 70,000 mg/Nm³, or more of tar, and the high quality DFB synthesis gas (i.e. exiting conditioner 210) comprises less than about 1 mg/Nm³ of tar. A frequent catalyst regeneration cycle through combustion reactor 235 (i.e. with a regeneration frequency in the approximate range of once every 10 seconds to 60 minutes) may contribute to maintaining catalyst activity under what could be considered severely coking conditions. (It is to be understood that circulation of catalytic heat transfer material about DFB conditioning loop 200 (and circulation of heat transfer material within a DFB gasification pyrolysis loop 100, when present, and as described hereinbelow) and thus regeneration thereof, is a substantially continuous process. In comparison, such severely coking conditions are generally impractical for nickel-based tubular steam methane reformers (SMRs) or fixed bed oxygen blown autothermal reformers (ATRs).

Depending on the application for which catalytic DFB conditioning loop 200 is utilized, suitable operating temperatures and pressures for conditioner/reformer 210 and combustor 235 may be determined as known in the art. In embodiments, conditioner/reformer 210 is operable at a temperature in the range of from about 1000° F. (538° C.) to about 2100° F. (1149° C.). In embodiments, conditioner 210 is operable at temperatures in the range of from about 1400° F. (760° C.) to about 1900° F. (1038° C.) or in the range of from about 1525° F. (829° C.) to about 1575° F. (857° C.). In some applications, conditioner/reformer 210 is operable at about 1550° F. (843° C.). Operation of the conditioner at a lower temperature may be desirable, although increased carbon dioxide carryover from combustor 235 may be obtained at lower temperatures. Operation of the conditioner at lower temperatures, in conjunction with reduced circulation rates of catalytic heat transfer material from combustor 235 via hot catalytic heat transfer line 215 may thus be desirable. Conditioner/reformer 210 may be configured for operation in the range of from about 2 psig (0.14 kg/cm²(g)) to about 1000 psig (70.3 kg/cm²(g)). Conditioner/reformer 210 may be configured for operation in the range of from about 2 psig (0.14 kg/cm²(g)) to about 5 psig (0.35 kg/cm²(g)). Conditioner/reformer 210 may be operable at or near ambient conditions. For example, conditioner/reformer 210 may be operable at about 2 psig (0.14 kg/cm²(g)). Alternatively, conditioner/reformer 210 may be operable at higher pressure, for example, a pressure in the range of from about 5 psig (0.35 kg/cm²(g)) to about 1000 psig (70.3 kg/cm²(g)).

Spent flue gas may exit combustion reactor 235 via spent flue gas outlet line 240. The spent flue gas in spent flue gas outlet line 240 may optionally have a temperature different than that of the flue gas with excess air introduced into combustor 235 via line 195. DFB product synthesis gas exits DFB conditioner/reformer 210 via DFB product gas outlet line 220.

Catalytic DFB 200 may be operable to upgrade or ‘condition’ synthesis gas from any source. For example, the DFB feedgas introduced into DFB 200 via line 150 may be or may comprise a crude low-quality synthesis gas. Such a crude synthesis gas may be obtained, for example, via gasification, reforming, and/or partial oxidation reactions. In embodiments, DFB feedgas comprises synthesis gas produced via gasification of a carbonaceous material. The crude low-quality synthesis gas may be obtained from gasification of a solid carbonaceous material including but not limited to coal, municipal sludge, petroleum coke, wellhead natural gas (which may be low quality), E-FUEL™, biomass, woody biomass refuse derived fuel (RDF), and combinations thereof. In embodiments, catalytic DFB 200 is operable to produce DFB product comprising synthesis gas, from a DFB feedgas that is primarily not synthesis gas, or that comprises substantial non-synthesis gas components. For example, in embodiments, the DFB feedgas comprises landfill gas, coal bed methane (CBM), coal mine methane (CMM), methanol purge gas (e.g. low pressure or ‘LP’ methanol purge gas), PSA tailgas, FT tailgas, refinery offgas, stranded gas from an oil well (e.g. a local oil well), coal oven gas, or some combination thereof. The DFB feedgas may comprise substantial amounts of carbon dioxide and methane and/or other hydrocarbons, that may be dry reformed within catalytic DFB 200 to produce additional (or any) synthesis gas. In embodiments, catalytic DFB 200 is operable with a feedgas comprising greater than or equal to 10, 20, 30, 40, 45, or 50 volume percent carbon dioxide. In embodiments, catalytic DFB 200 is operable with a feedgas comprising greater than or equal to 10, 20, 30, 40, 45, or 50 volume percent methane and/or other hydrocarbons. In embodiments, catalytic DFB 200 is operable with a feedgas comprising less than or equal to 50, 40, 30, 20, or 10 volume percent carbon monoxide. In embodiments, catalytic DFB 200 is operable with a feedgas comprising less than or equal to 50, 40, 30, 20, or 10 volume percent hydrogen. In embodiments, catalytic DFB 200 is operable with a feedgas comprising greater than, less than, or equal to 10, 20, 30, 40, 50, 60, 70, 80, 90, or 100 volume percent synthesis gas (i.e. hydrogen+carbon monoxide).

FT Synthesis Apparatus.

The system of this disclosure further comprises FT synthesis apparatus 45. FT synthesis apparatus 45 comprises at least one FT synthesis reactor 20, and at least one product separator 40. FT synthesis reactor 20 is configured to produce FT hydrocarbons from an FT syngas feed comprising at least a portion of the synthesis gas in the DFB product gas. In applications, the integration of catalytic DFB 200 with FT processing is utilized to provide an FT feedgas of a desired mole ratio of hydrogen to carbon monoxide and/or a desired purity for use in Fischer-Tropsch conversion. In embodiments, the one or more Fischer-Tropsch reactors is operable with an iron-based FT catalyst. In embodiments, the one or more Fischer-Tropsch reactors is operable with an cobalt-based FT catalyst. In embodiments, the iron-based Fischer-Tropsch catalyst is a precipitated unsupported catalyst. In embodiments, the Fischer-Tropsch catalyst is a catalyst as disclosed in U.S. Pat. No. 5,504,118, U.S. patent application Ser. No. 12/198,459, and/or U.S. patent application Ser. No. 12/207,859, each of which is hereby incorporated herein by reference in its entirety for all purposes not contrary to this disclosure. FT feedgas is introduced into FT reactor 20 via FT feedgas inlet line 15. FT reactor 20 is configured to provide a gaseous FT product or overhead, and a liquid FT product comprising molten FT wax. The FT overhead generally comprises volatilized LFTL, carbon dioxide, methane, and unreacted carbon monoxide and hydrogen. The liquid FT product may comprise primarily C5+ hydrocarbons. An FT overhead outlet line 26 is configured to extract FT overhead from FT reactor 20. An FT product line 25 is configured to extract FT liquid product from FT reactor 20.

FT synthesis apparatus 45 further comprises product separator 40, fluidly connected with FT reactor 20. Product separator 40 is configured to separate the FT overhead into an LFTL tailgas and an LFTL liquid product comprising LFTL. Product separator 40 may be an apparatus selected from distillation columns. In embodiments, the FT tailgas comprises light hydrocarbons (e.g. methane, ethane, propane, light oxygenates (e.g. C1-C3), light olefins (e.g. C2-C3)), carbon dioxide, nitrogen, and unreacted hydrogen and carbon monoxide. The molar ratio of hydrogen to carbon monoxide in the FT tailgas may be higher than that of the desired FT feedgas. An FT tailgas outlet line 46 may be configured to extract FT tailgas from product separator 40, and a LFTL product line 48 may be configured to extract LFTL separated from the FT overhead within product separator 40.

FT synthesis apparatus 45 may further comprise syngas conditioning apparatus 10. Syngas conditioning apparatus 10 is configured to condition synthesis gas prior to introduction thereto into FT reactor 20. Syngas conditioning apparatus 10 may be operable to remove one or more undesirable components from synthesis gas, prior to introduction thereto into FT reactor 40. Syngas conditioning apparatus 10 may be operable to remove one or more components selected from sulfur (and sulfur-containing compounds, such as, but not limited to hydrogen sulfide), carbon dioxide, and tar from at least a portion of the synthesis gas being introduced into FT reactor 20. Syngas conditioning apparatus 10 may comprise one or more units selected from sulfur removal units, carbon dioxide removal units, and tar removal units. In embodiments, syngas conditioning apparatus 10 comprises no tar removal apparatus. In embodiments, syngas conditioning apparatus comprises at least one tar, carbon dioxide, aromatic, and/or hydrogen sulfide removal unit. Such units may be selected from absorbers, membranes, OLGA units, DAHLMANN units, acid gas removal units, and the like. In embodiments, syngas conditioning apparatus 10 comprises one or more caustic scrubbers. The caustic scrubber(s) may be adapted for removing substantially all of any residual low levels of carbonyl sulfide and/or other acid gases such as H₂S from the high-quality synthesis gas extracted from catalytic DFB 200 via DFB product outlet line 220. In embodiments, syngas conditioning apparatus 10 is configured to remove more than 99.9 volume percent of the carbonyl sulfide or other acid gas(es) in the DFB product, to provide a scrubbed high-quality synthesis gas. In embodiments, syngas conditioning apparatus 10 comprises a ZnO polishing bed configured for the removal of residual H₂S. Syngas conditioning apparatus 10 may be fluidly connected with FT reactor 20 via FT feedgas inlet line 15. It is to be understood that catalytic (e.g. Ni) DFB unit 200 may render such aforementioned conventional conditioning unit(s) (e.g. tar removal unit(s)) unnecessary for normal operation. For certain cobalt-based FT processes, supplemental, conventional CO₂ removal unit(s) may be utilized in addition to the carbon dioxide removal provided by the catalytic DFB.

FT synthesis apparatus 45 may further comprise an overhead heat recovery apparatus 30. Overhead heat recovery unit or apparatus 30 may be positioned downstream of and fluidly connected with FT reactor 20, and upstream of and fluidly connected with product separator 40. Overhead heat recovery apparatus 30 may be any suitable heat recovery apparatus known in the art. FT reactor overhead outlet line 26 may fluidly connect FT reactor 20 with overhead heat recovery apparatus 30. Overhead heat recovery outlet line 35 may fluidly connect overhead heat recovery apparatus 30 with product separator 40.

FT synthesis apparatus 45 may further comprise product upgrading apparatus 50. Product upgrading apparatus 50 is fluidly connected with product separator 40 and/or with FT reactor 20, such that at least a portion of the LFTL, at least a portion of the liquid FT product, or at least a portion of both the LFTL and the liquid FT product may be introduced thereto. For example, LFTL product line 48 may fluidly connect product separator 40 with product upgrading apparatus 50, such that at least a portion of the LFTL may be introduced thereto. FT product line 25 may fluidly connect FT reactor 20 with product upgrading apparatus 50, such that at least a portion of the FT liquid product (i.e. molten FT wax) may be introduced thereto. Product upgrader apparatus 50 may comprise any suitable upgrading apparatus known in the art. For example, product upgrading apparatus 50 may comprise one or more units selected from hydroisomerizers, hydrocrackers, hydrotreaters, distillation columns, and combinations thereof. In embodiments, product upgrading apparatus 50 comprises one or more hydro-processing units, such as, but not limited to hydroisomerizers, hydrocrackers, and hydrotreaters. One or more lines 55 may be configured to extract upgraded FT product from product upgrading apparatus 50. Such upgraded FT product generally comprises one or more synthetic fuel. Such synthetic fuels include, but are not limited to, FT naphtha, FT diesel, FT gasoline, and FT jet fuel. In embodiments, the synthetic fuel comprises FT naphtha. In embodiments, the synthetic fuel comprises FT diesel. In embodiments, the synthetic fuel comprises FT gasoline. In embodiments, the synthetic fuel comprises FT jet fuel.

Gasification Apparatus 100.

As noted hereinabove, the disclosed system may further comprise synthesis gas generation apparatus configured to produce synthesis gas for use as a component (or the entirety) of the DFB feedgas, and/or for use as a component of the FT feedgas. Any suitable synthesis gas generation apparatus known in the art may be utilized. In embodiments, the synthesis gas generation apparatus comprises one or more apparatus selected from gasification apparatus, reforming apparatus, and partial oxidation apparatus. In embodiments, the disclosed system comprises gasification apparatus 100. In embodiments, gasification apparatus 100 is an indirect gasification apparatus (i.e. non-air or oxygen blown). Any suitable indirect gasification apparatus may be employed. In embodiments, a ClearFuels or SilvaGas type gasification apparatus is employed.

As indicated in the embodiment of FIG. 1, gasification apparatus 100 may be positioned upstream of catalytic DFB 200, and/or upstream of FT synthesis apparatus 45. In embodiments, gasification apparatus 100 is fluidly connected with DFB 200 via catalytic DFB feedgas inlet line 150 (and optionally solid/gas separator 400A, which is further described hereinbelow). Gasification apparatus 100 comprises gasifier 140, and may further comprise a carbonaceous material handling/preparation apparatus 90.

Dual Fluidized Bed Gasifier.

In embodiments, the herein disclosed synthetic hydrocarbons and/or synthetic fuels production system comprises a dual fluidized bed gasifier, as described U.S. patent application Ser. No. 12/691,297, filed Jan. 21, 2010, and now U.S. Pat. No. 8,241,523. A suitable dual fluidized bed gasifier will now be described with reference to FIG. 5. In embodiments, the herein disclosed system comprises apparatus for producing low-quality synthesis gas or producer gas for introduction into dual fluid bed reformer loop 200 (via line 150) as at least a portion of the DFB feedgas. As shown in the embodiment of FIG. 5, in embodiments, the herein disclosed system further comprises a dual fluid bed loop 100, which is a gasification pyrolysis loop for producing product gas comprising low-quality synthesis gas. In embodiments, dual fluid bed loop 100 is a primary gasification loop and dual fluid conditioning loop 200 is downstream thereto, i.e. is a secondary loop, and may be referred to herein as a secondary conditioning loop or a secondary reforming loop. Dual fluid bed gasification loop 100 comprises fluid bed gasifier 140 fluidly connected to combustion reactor 185 via ‘cold’ bed material circulation line 145 and ‘hot’ bed material circulation line 155.

Gasifier 140 is any fluid bed gasifier suitable for the gasification of a carbonaceous feed material to form a producer gas comprising synthesis gas. Gasifier 140 may contain, and circulated about primary gasification/pyrolysis loop 100 may be, a bed of heat transfer material selected from silica, olivine, alumina (e.g. alpha-alumina, γ-alumina, etc.), other suitable attrition resistant materials, and combinations thereof. In embodiments, the heat transfer material of DFB gasification loop 100 comprises silica. In embodiments, the heat transfer material of DFB gasification loop 100 comprises alumina. In embodiments, the heat transfer material of DFB gasification loop 100 comprises olivine. Utilization of heat transfer material such as silica may enable operation of dual fluid bed gasification loop 100 at high temperature. Bed material may be introduced wherever suitable, for example, a line 190 may be used to introduce makeup bed material to combustion reactor 185. In this manner, undesirables, if present, may be removed from the bed material via combustion. As with combustion reactor 235, combustion reactor 185 may be oxygen-blown or air-blown.

In embodiments, combustion reactor 185 and/or combustion reactor 235 are air-blown, and no air separation unit is required to separate oxygen from air for use as oxidant in the combustor(s). In embodiments, a steam inlet line 135 and a carbonaceous feed inlet line 125 are configured to introduce steam (e.g. low pressure steam) and carbonaceous feed material, respectively, into gasifier 140.

Rather than, or in addition to, introduction into reformer 210 via recycle tail gas reactant line 46A′, recycle tail gas from downstream Fischer-Tropsch synthesis apparatus 45, methanol production, or other downstream chemical synthesis operations in downstream processing unit(s) may be used in place of at least a portion of the low pressure steam in line 135 to a partial or complete extent as required for gasification fluidization velocity requirements as long as sufficient moisture is present in the feedstock in line 125 for gasification and conditioning/reforming purposes. While reducing costly steam consumption, such tailgas recycle could, in applications, be used to minimize associated downstream waste water production. Using this recycle tail gas as a fluidizing transport medium for solid feeds in place of steam could also apply in a similar capacity to other indirect gasification technologies based, for example, on stationary tubular heat transfer media. In embodiments, such an alternative indirect gasification technology is used in place of a primary pyrolysis loop 100 to provide low grade producer gas comprising synthesis gas for introduction, via line 150, into catalytic DFB 200 as at least a component of the DFB feedgas.

In embodiments, an inlet line 130 may connect gasifier 140 with a source of liquid or high sulfur vapor hydrocarbons. Gasifier 140 is operable to convert carbonaceous feed material and optionally liquid or high sulfur vapor hydrocarbons into product gasification or producer gas comprising synthesis gas, at least a portion of which may be conditioned in conditioner 210 of catalytic DFB loop 200. A product outlet line 150 may fluidly connect gasifier 140 of primary gasification loop 100 with conditioner 210 of catalytic DFB loop 200. Thus, line 150 may be configured for introduction of gasification product gas comprising low-quality synthesis gas (i.e. producer gas) from any suitable source into conditioner 210. Alternatively, or additionally, line 150 may introduce non-synthesis gas or non-syngas components into catalytic DFB 200, as further described hereinbelow with reference to the embodiment of FIG. 4.

As gasification requires heat, ‘cold’ bed material circulation line 145 connects gasifier 140 with combustion reactor 185, whereby a portion of the bed material in gasifier 140 is introduced from gasifier 140 into combustion reactor 185. Combustion reactor 185 is operable such that any unconverted char and ash in the circulated ‘cold’ bed material (e.g. ‘cold’ silica) is combusted. Combustion reactor 185 is any combustor suitable for the combustion of unconverted material including char and ash into flue gas in the presence of oxidant and fuel. A flue gas outlet line 195 fluidly connects combustion reactor 185 of primary gasification dual fluid bed loop 100 with combustion reactor 235 of secondary conditioning DFB loop 200.

Oxidant inlet line 175 and fuel inlet line 180 are connected to combustion reactor 185 for the respective introduction of oxidant and fuel thereto. As mentioned hereinabove with respect to fuel line 230, the fuel may comprise tailgas purge from a Fischer-Tropsch reactor and fuel line 180 may be fluidly connected with a tailgas outlet line 46/46A/46A″ of a Fischer-Tropsch reactor and/or product separator 40 of FT synthesis apparatus 45. The oxidant introduced via oxidant inlet line 175 may be substantially-pure oxygen, however air is desirably utilized as oxidant. In such applications, no air separation unit or expensive substantially-pure oxygen may need to be employed. As indicated in FIG. 5, a line 250 may be utilized to provide oxidant (e.g. air, oxygen, or substantially-pure oxygen) from oxidant inlet line 175 to combustor 235 of conditioning loop 200. However, it may be desirable to pass the oxidant (e.g. air) required in combustor 235 through combustor 185, such that combustor 185 may be operated at a lower temperature by virtue of the resulting higher oxygen partial pressure, thus reducing the likelihood of the production of undesirables, such as dioxin, NOₓ, and etc., as well as reducing the likelihood of melting of ash constituents and the associated agglomeration and volatilization. Gas turbine exhaust in line 255 comprising substantial oxygen and optionally at elevated temperature may, in embodiments, be introduced into combustor 185 and/or combustor 235 via lines 260 and 265 respectively. Utilization of gas turbine exhaust within combustor 235 and/or combustor 185 may reduce the size required compression requirements.

‘Hot’ bed material circulation line 155 connects combustion reactor 185 with gasifier 140, such that heated bed material from which undesirable ash, tar and/or other combustible material has been removed (e.g. ‘hot’ silica) may be circulated back into gasifier 140. A purge line 160 may be configured to purge unwanted components from primary gasification loop 100 of system 10. Such unwanted components may comprise, for example, ash, sulfate, chloride, or some combination thereof.

Depending on the feed material introduced via carbonaceous feed inlet line 125, gasification apparatus 100 may be configured for the removal of sulfur, halides, or other contaminants from the product gas. For example, a line 190 may be configured for the introduction of at least one compound into combustion reactor 185. The at least one component may be selected from calcium oxide (lime), magnesium oxide, sodium carbonate, sodium bicarbonate and other alkalis. Suitable metals, such as an iron catalyst slurry wax purge produced from a slurry phase Fischer-Tropsch reactor of FT synthesis apparatus 45, may also be introduced, for example, via line 130, via line 190, or both. While the wax content of the catalyst wax slurry will be pyrolyzed in gasification unit 140, the iron content of the slurry may also contribute to the removal of sulfur, chlorides, and/or other undesirables from the product syngas via, for example, purge extraction via purge line 160 and/or along with spent flue gas in line 240. Addition of spent iron FT catalyst from a FT reactor(s) of FT synthesis apparatus 45 to the combustor may promote formation of iron oxides (e.g. Fe₂O₅) which may react with alkali salts to form XeFe₂O₄, which melts at a higher temperature (about 1135° C.), helping to prevent agglomeration. Other additives, such as, but not limited to, limestone, alumina, and dolomite may also aid in a similar fashion, by providing a higher melting point eutectics (which may, in embodiments, be less than 1135° C.).

In applications, gasifier 140 of gasification DFB loop 100 operates at a lower temperature than reformer 210 of catalytic DFB conditioning loop 200. In applications, gasifier 140 is operable at a temperature in the range of from about 1100° F. (593° C.) to about 1700° F. (927° C.); alternatively in the range of from about 1200° F. (649° C.) to about 1600° F. (871° C.); alternatively about 1300° F. (704° C.). The generally lower operational range permitted for gasification pyrolysis loop 100 may help to promote contaminant capture in purge stream 160 and/or increase the thermal efficiency of the pyrolysis/gasification. The lower operating temperatures and aforementioned sorbent addition suitable for use in primary loop 100 also minimize formation of dioxin and thermal NOx in the flue gas stream exiting primary combustor 185 via line 195. Such lower temperature operation also reduces volatilization of alkali halide salts and eutectic mixtures, which may reduce/prevent deactivation of catalyst in catalytic DFB loop 200 and fouling and/or corrosion of downstream equipment. Such lower temperature operation may be particularly advantageous when the aforementioned tubular gasification apparatus or other gasification apparatus is used in place of the dual fluid bed gasification loop 100 depicted in the embodiment of FIG. 5. The capability of DFB gasification loop 100 to operate at lower temperatures for the production of the low quality syngas to be introduced into conditioner 210 via line 150 may reduce the thermal heat transfer duty, metallurgical stresses, and/or the operational severity for such gasifiers (e.g. tubular gasifiers) while similarly improving overall yields of high quality syngas facilitated by shifting at least a fraction of the gasification/reforming duty to conditioning loop 200. The resulting yield of FT liquids may increase by over 30%, 40%, 50% or more relative to a base case with an indirect tubular gasifier without the proposed conditioner/reformer. A substantial capital cost reduction for such gasifiers (e.g. tubular gasifiers) may thus also result when integrated in this manner with DFB conditioning loop 200.

As indicated by the dashed lines connecting the primary and secondary DFB loops in FIG. 5, in embodiments, the primary and secondary units can also be structurally integrated to further reduce costs. In this manner, the shells of the secondary units (i.e. conditioner 210 and combustor 235) may be mounted on top of the shells of the corresponding primary units (i.e. gasifier 140 and combustor 185). For example, in embodiments, primary gasifier 140 may be structurally integrated with secondary conditioner/reformer 210; primary combustion unit 185 may be structurally integrated with secondary combustion unit 235; or both pairs of units may be structurally integrated, as depicted via dashed lines in the embodiment of FIG. 5.

While an embodiment of the disclosed system comprising a dual fluid bed conditioning/reformer loop 200 downstream of a dual fluid bed gasifier loop 100 has been described in detail herein, similar high thermal efficiency may be obtained with a gasification product gas provided via a gasifier operable by an other type of ‘indirect’ gasification technology in which air is indirectly used as a gasification (combustion) agent without diluting the product synthesis gas with the nitrogen content of the air and resulting flue gas. Gasifiers operating via various indirect gasification technologies may be integrated with conditioning, catalytic DFB loop 200. Yield improvement (e.g. 30% or greater) in terms of high quality syngas production and the resulting increased FT product yields may result when a conditioning DFB loop 200 as disclosed herein is applied to synthesis gas and flue gas effluents from these various technologies. In this manner, similar yield improvement to that provided by integration of DFB conditioning loop 200 with DFB gasification loop 100, as depicted in the embodiment of FIG. 5 and described in detail herein, may be effected. Integration of DFB reformer loop 200 as disclosed with various indirect gasification technologies may enable the use of gasification feeds containing higher amounts of sulfur via addition of a desulfurizing agent (e.g. a lime-based desulfurization agent) to a (e.g. fluid bed) gasifier. For example, in embodiments, the feed to gasifier 140 may comprise more than 0.5, 5.0, or 10.0 weight percent sulfur in embodiments.

Similarly, the dual fluid bed conditioning loop 200 of this disclosure may be integrated with a gasifier operating via more conventional ‘direct’ gasification technology for the similar purpose of upgrading the quality (i.e. conditioning) the synthesis gas produced, as long as the low quality synthesis gas (for introduction into DFB loop 200 via line 150 in the embodiment of FIG. 5) has a sufficiently low sulfur content. Such gasifiers based on fluid beds may be integrated with a dual fluid bed reformer loop 200 of this disclosure allowing gasification of higher sulfur feedstocks (introduced thereto via line 125 in FIG. 5) via addition of a desulfurizing agent (e.g. a lime-based desulfurizing agent) to the gasifier.

Carbonaceous Material Handling Apparatus.

As noted hereinabove, the herein disclosed system may further comprise carbonaceous feed handling apparatus 90, associated with gasification apparatus 100. A line 85 may be configured to introduce carbonaceous feed material to carbonaceous feed handling apparatus 90. Carbonaceous feed handling apparatus 90 may be fluidly connected with gasifier 140 via carbonaceous feed inlet line 125. Any suitable feed handling apparatus known in the art may be employed. For example, feedstock handling apparatus 90 may comprise a collection bin and a screw feeder connected via a screw feeder inlet line, one or more dryers, or a combination thereof. A bulk feed inlet line may be adapted for introduction of bulk carbonaceous feed into the solid feedstock collection bin. The solid feedstock collection bin may be a funnel-shaped unit. The screw feeder line may be configured for introduction of collected feed into the screw feeder. The screw feeder may be adapted for introduction of carbonaceous feed material into gasifier 140 via carbonaceous feed inlet line 125.

Heat Recovery Unit.

As noted hereinabove, and depicted in FIGS. 1, 2, and 4, a system of this disclosure may comprise one or more heat recovery units configured to extract heat from the DFB product gas in line 220. Any suitable heat recovery apparatus known in the art may be utilized.

Gas/Solid Separation Units.

In embodiments, separation of bed material from the reactor overheads of conditioner/reformer 210, combustor 235 and, when present, from gasification reactor 140, and combustor 185 is provided by suitable gas/solid separation units. Thus, in applications, the herein disclosed system may comprise at least one, at least two, at least three or at least four gas/solids separation units. Such gas/solids separation units may be positioned on bed material transfer lines 225, 215, 145, 155, or a combination thereof. In embodiments, the system comprises one or a plurality of cyclones to effect gas/solid separation. In applications, a candle filter(s) is (are) used rather than or in series with a cyclone(s). Candle filters may be capable of a finer degree of particle separation (although this may be unnecessary in embodiments) and may also have a lower height requirement than cyclones, thereby possibly minimizing the height requirements of the various reactors (i.e. 210, 235, 140 and/or 185).

In embodiments, the herein disclosed system comprises a solid/gas separator 400A, positioned upstream of catalytic DFB 200, a solids/gas separator 400B, positioned downstream of catalytic DFB 200, or both. As indicated in FIGS. 1, 2, and 4, a solids/gas separation apparatus 400A may be fluidly connected with gasification apparatus 100, whereby gasifier product gas may be introduced thereto via line 150, prior to introduction of the solids-reduced gas into catalytic DFB 200. Solids may be extracted from gas/solids separation apparatus 400A via solids outlet line 405A. A solids/gas separation apparatus 400B may be fluidly connected with catalytic DFB apparatus 200, whereby DFB product gas may be introduced thereto, for example, via line 505 (optionally subsequent heat recovery via heat recovery apparatus 500). Solids may be extracted from gas/solids separation apparatus 400B via solids outlet line 405B.

In embodiments in which DFB gasification loop 100 produces low-quality synthesis gas to be conditioned in catalytic DFB loop 200, a gas/solids separation unit 400A may be positioned between the gasification loop 100 and DFB conditioning/reforming loop 200. The gas/solid separation unit may be any effective solid/gas separation device known in the art. For example, suitable devices include, but are not limited to, cyclones, filters and candle filters. In embodiments, conventional candle filters are used as the one or more gas/solids separation devices associated with the gasifier and/or combustor of gasification loop 100. When candle filters are utilized, contaminant removal agents (e.g. sulfur and/or halide removal agents) may tend to form a temporary layer on the surface of the filters.

Compressors.

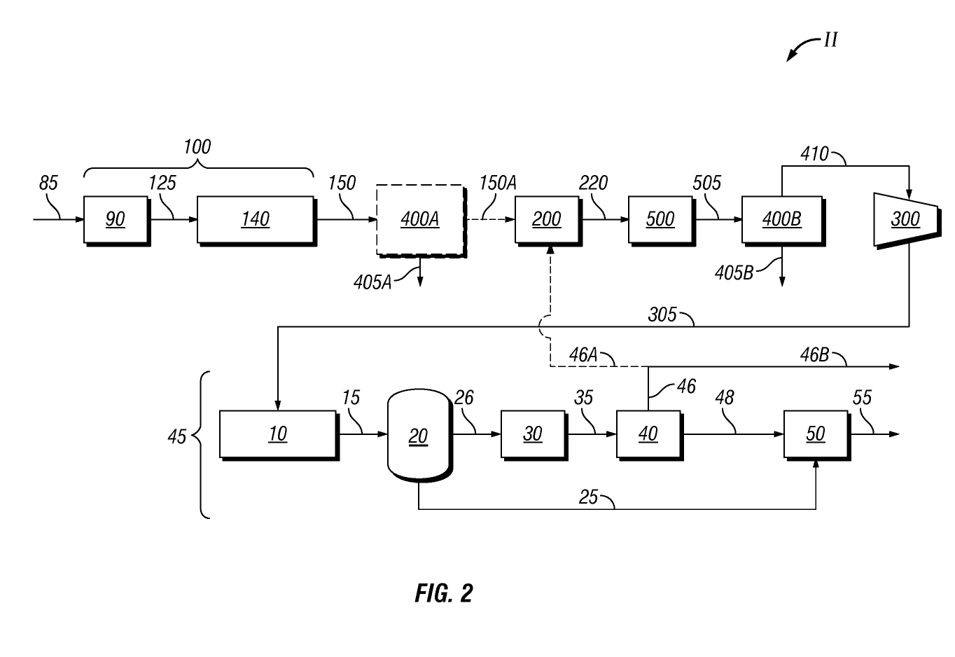

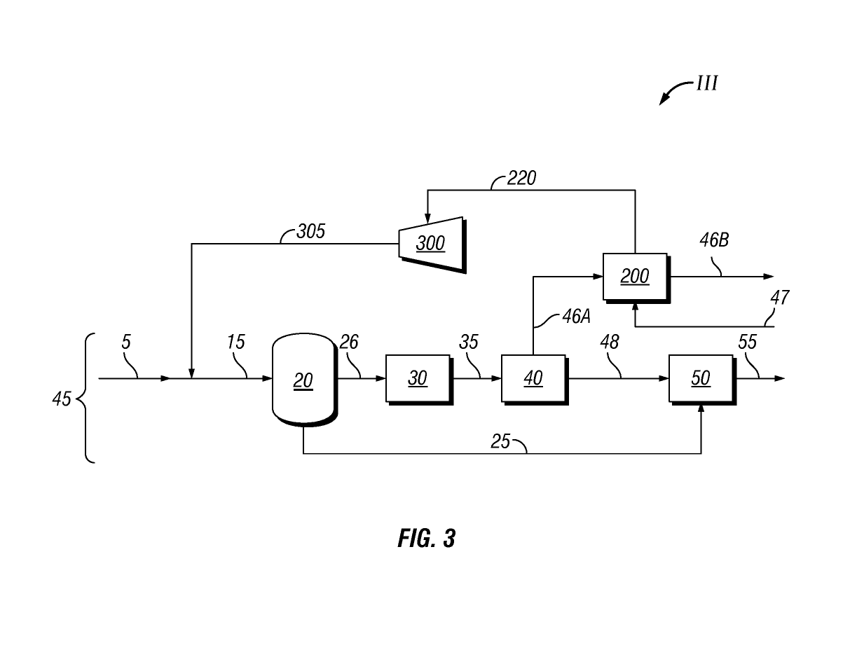

The herein disclosed system may comprise a compressor 300 downstream of catalytic DFB 200, and configured to increase the pressure of a synthesis gas containing stream prior to introduction into FT synthesis apparatus 45. Compressor 300 may be downstream of heat recovery apparatus 500, gas/solids separation apparatus 400B, or downstream of both. In the embodiment of FIGS. 1 and 2, compressor 300 is downstream of gas/solids separation apparatus 400B and heat recovery apparatus 500, and a line 410 is configured to introduce solids-reduced DFB product gas comprising synthesis gas into compressor 300. In the embodiment of FIG. 3, compressor 300 is downstream of catalytic DFB apparatus 200, and DFB outlet line 220 is configured to introduce DFB product gas comprising synthesis gas into compressor 300. In the embodiment of FIG. 4, compressor 300 is downstream of heat recovery apparatus 500, and line 410 is configured to introduce temperature-reduced DFB product gas comprising synthesis gas into compressor 300. A compressed DFB product line 305 may fluidly connect compressor 300 with FT synthesis apparatus, for example, may fluidly connect compressor 300 with syngas conditioning apparatus 10, as in the embodiments of FIGS. 1, 2, and 4. Compressed DFB product line 305 may fluidly connect compressor 300 with FT reactor feedgas line 15, as in the embodiment of FIG. 3.

FT Tailgas Recycle Line.

As mentioned hereinabove, in embodiments, the system of this disclosure further comprises an FT tailgas recycle line 46A configured to introduce at least a portion of the FT tailgas extracted from FT synthesis apparatus 45 into catalytic DFB 200. Dry reforming of the non-synthesis gas components of the FT tailgas via introduction thereof, as a feed component (or as the entirety of the DFB feedgas, as discussed hereinbelow with reference to embodiment of FIG. 3), into catalytic DFB via FT syngas recycle line 46A, may increase the overall synthetic fuel yield of the plant (e.g. may increase the pounds per day (or PPD) of synthetic fuels produced per pound of biomass feed).

As indicated in the embodiment of FIG. 2, an FT tailgas recycle line 46A may be configured to introduce at least a portion of the FT tailgas extracted from product separator 40 via FT tailgas outlet line 46 into catalytic DFB 200. FT tailgas recycle line 46A may be configured to introduce at least a portion of the FT tailgas into catalytic DFB 200 as a feed and/or as a fuel. For example, with reference to FIG. 5, an FT tailgas recycle line 46A″ may be configured to introduce FT tailgas into combustor 235, and/or an FT tailgas line 46A′ may be configured to introduce FT tailgas as a feed component (optionally in addition to gasification product gas in line 150) into conditioner 210. That is, FT tailgas line 46A may be connected with (or may be the same as) fuel line 230, may be connected with (or may be the same as) line 205 and/or 150, or FT tailgas line 46A may be connected with some combination of fuel line 230, feed line 205, and DFB feedgas line 150.

As indicated in the embodiment of FIG. 3, and further described hereinbelow, the disclosed system may be configured such that FT tailgas recycle line 46A provides substantially all of the DFB feedgas to catalytic DFB 200. In other embodiments, the system is configured such that FT tailgas in FT tailgas recycle line 46A is combined with additional gas to provide the DFB feedgas. For example, in the embodiment of FIG. 2, at least a portion of the FT tailgas may be introduced as a component of the DFB feedgas, along with gasification product gas in line 150. In embodiments, such as that of FIG. 4, which is further described hereinbelow, the system is configured such that the FT tailgas in FT tailgas recycle line 46A is combined with low or medium BTU fuel gas in non-synthesis gas DFB inlet gas line 270, to provide the DFB feedgas. In such embodiments, the system is configured for operation with a DFB feedgas that is not primarily synthesis gas. In this embodiment, a bypass line 270 may be configured to introduce a portion of the non-synthesis gas into DFB 200 as a fuel (e.g. via line 230 into combustor 235 of FIG. 5).

Method.

Also disclosed herein is a method of producing synthetic hydrocarbons and/or synthetic fuels. The disclosed method comprises producing a DFB product from a DFB feedgas, via a catalytic dual fluidized bed (DFB), wherein the DFB product comprises synthesis gas, and introducing an FT feedgas comprising at least a portion of the DFB product into an FT synthesis reactor, and extracting a gaseous FT overhead and a liquid FT product comprising FT wax from the FT reactor. The method may further comprise separating, from the FT overhead, an FT tailgas and an LFTL product comprising LFTL, and/or upgrading at least a portion of the LFTL product, at least a portion of the liquid FT product, or at least a portion of both the LFTL product and the liquid FT product, thus providing one or more synthetic fuels. In embodiments, the method further comprises introducing at least a portion of the FT tailgas into the catalytic DFB. The FT tailgas may be introduced into the catalytic DFB as a fuel, as at least a component of the DFB feedgas, or both. In embodiments, the DFB feedgas consists primarily FT tailgas. In embodiments, the DFB feedgas comprises primarily non-synthesis gas. For example, in embodiments, the DFB feedgas comprises primarily low and/or medium BTU fuel gas, FT tailgas, or a combination thereof. The various embodiments of the herein disclosed method will be described in more detail hereinbelow.

Embodiments of the herein disclosed method will now be described with reference to the Figures. According to the embodiments of FIGS. 1 and 2, DFB feedgas is introduced into catalytic DFB 200, via line 150. Catalytic DFB 200 may be operated via any means known in the art to convert non-synthesis gas components of the DFB feedgas into additional synthesis gas. A method of operating a suitable catalytic DFB is detailed hereinbelow. At least a portion of the DFB feedgas may be produced via gasification (e.g. of coal and/or biomass), via reforming (e.g. natural gas reforming), and/or via partial oxidation. In the embodiment of FIGS. 1 and 2, respectively, all or a portion of the DFB feedgas is produced via gasification in gasification apparatus 100. Such production of synthesis gas via gasification may be effected via any means known in the art. A suitable method for the production of synthesis gas via DFB gasification of a carbonaceous feed material will be detailed hereinbelow.

As mentioned hereinabove, in embodiments, the DFB feedgas comprises FT tailgas. In embodiments, the DFB feedgas comprises greater than or equal to about 10 volume percent, 20 volume percent, 30 volume percent, 40 volume percent, 50 volume percent, 60 volume percent, 70 volume percent, 80 volume percent, 90 volume percent, or 100 volume percent FT tailgas. For example, as indicated in the embodiments of FIG. 1-3, the DFB feedgas may comprise from about 0 to about 100 volume percent synthesis gas (e.g. produced via gasification, reforming, and/or partial oxidation), from about 10 to about 90 volume percent synthesis gas, from about 20 to about 80 volume percent synthesis gas, from about 30 to about 70 volume percent synthesis gas, from about 40 to about 60 volume percent synthesis gas, or from about 45 to about 55 volume percent synthesis gas; and from about 100 to about 0 volume percent FT tailgas, from about 90 to about 10 volume percent FT tailgas, from about 80 to about 20 volume percent FT tailgas, from about 70 to about 30 volume percent FT tailgas, from about 60 to about 40 volume percent FT tailgas, or from about 55 to about 45 volume percent FT tailgas.

The amount of FT tailgas introduced into catalytic DFB 200 as feed or fuel may depend on the fuel duty of the catalytic DFB, the syngas requirements of the system, the feed to an upstream gasifier 140 of gasification apparatus 100, etc. The recycle of FT tailgas is facilitated by the low pressure operation of the catalytic DFB.

In embodiments, the DFB feedgas comprises a gas other than synthesis gas. In embodiments, the DFB feedgas does not comprise synthesis gas produced via gasification, reforming, or partial oxidation. In embodiments, the DFB feedgas comprises FT tailgas, low BTU fuel gas, medium BTU fuel gas, or a combination thereof. In embodiments, the DFB feedgas comprises primarily, or consists essentially of FT tailgas. For example, in the embodiment of FIG. 3, the DFB feedgas comprises FT tailgas.

In embodiments, the DFB feedgas comprises low and/or medium BTU fuel gas. In embodiments, the DFB feedgas comprises primarily, or consists essentially of, low and/or medium BTU fuel gas. For example, in the embodiment of FIG. 4, the DFB feedgas comprises fuel gas (e.g. low and/or medium BTU fuel gas), and may also comprise FT tailgas. In embodiments, the fuel gas utilized as at least a component of the DFB feedgas comprises hydrocarbons and carbon monoxide, which may be dry reformed within catalytic DFB 200, to provide synthesis gas. Such fuel gas may be selected from low and medium BTU fuel gas. For example, such fuel gas may include, but is not limited to, coal bed methane (CBM), coal mine methane (CMM), landfill gas, flare offgas, methanol purge loop gas, PSA tailgas, FT tailgas, flare gas, and/or stranded gas from an oil well (e.g. localized stranded gas from a local oil well). For example, coke oven gas may be available in applications in which ethanol production is incorporated downstream. As noted hereinabove, the conversion of such fuel gas into synthesis gas, and thus into FT product, via dry reforming of the hydrocarbons and carbon dioxide in the fuel gas may reduce carbon dioxide emissions typically associated with the disposal of such fuel gas. One of the attractions of such a use of fuel gas is that little or no preparation thereof may be required prior to introduction into catalytic DFB 200. Furthermore, such fuel gas usage may increase the thermal efficiency of conversion technology with little or no usage of steam, in contrast to conventional methods, such as SMR discussed hereinabove.

Within catalytic DFB 200, non-synthesis gas components of the DFB feedgas are converted into synthesis gas. For example, tar and carbon dioxide in the DFB feedgas may be dry reformed into synthesis gas. Such integration of catalytic DFB with downstream FT synthesis may eliminate or reduce the extent of other cleanup and/or conditioning operations upstream of FT synthesis. For example, dry reforming of tar and/or CO₂ may reduce or eliminate the need for tar removal, carbon dioxide removal, or both, upstream of FT reactor 20. The incorporation of catalytic dual fluidized bed reforming may increase the overall efficiency of synthetic fuels production. For example, when biomass gasification is utilized to provide at least a portion of the DFB feedgas, incorporation of catalytic DFB reforming of the gasification product gas upstream of FT synthesis may increase the overall conversion of biomass to synthesis gas, and thus may also increase the overall conversion of biomass to synthetic fuel.

Fuel gas for usage as at least a component of the DFB feedgas is available from a number of industries. The composition and operating conditions of available fuel gas will, of course, vary with source, however, example fuel gases and expected compositions and operating conditions are provided in Table 1.

TABLE 1
Exemplary Fuel Gas Suitable for use in DFB Feedgas

| Fuel Gas | Pressure | Temperature | Exemplary Composition |
|---|---|---|---|
| Landfill Gas (LFG) | Atmospheric | Ambient | 50% CO₂, 50% CH₄, trace |
| | | | components (e.g. S) |
| Coal Bed Methane or Coal | Atmospheric | Ambient | 50% CO₂, 50% CH₄, trace |
| Mined Methane (CBM or | | | components (e.g. S) |
| CMM) | | | |
| LP Methanol Purge Gas | ~300 psig | ~100° F. | 47% H₂, 36% CH₄, 10% CO₂, |
| | | | 3% CO, 2% CH₃OH |
| PSA Tailgas | ~5 psig | Ambient | 55% CO₂, 25% H₂, 20% CH₄, |
| | | | trace components (e.g. S) |
| FT Tailgas | ~300 psig | ~60° F. | 40% CO₂, balance CH₄ & Other |
| | | | Light HC's |
| Refinery Offgas | ~300 | Ambient | 30% CO₂, balance CH₄ & H₂ |
| Stranded Gas from Oil | N/A | Ambient | Mixtures of CO₂, CH₄, and Light |
| Well | | | HC's |
| Coke Oven Gas | Varies | Varies | Varies |

Following catalytic DFB conditioning, at least a portion of the DFB product gas is introduced into FT production apparatus 45. As indicated in the embodiment of FIG. 1, the DFB product gas may be introduced into heat recovery apparatus 500 via DFB product gas outlet line 220.

Solids may be removed from the DFB product gas via gas/solids separation apparatus 400B, and solids removal line 405B. For example, heat reduced DFB product gas may be introduced via line 505 into gas/solids separation apparatus 400B, and solids-reduced DFB product gas extracted via line 410. The pressure of the DFB product gas may be increased to a pressure desirable for FT synthesis. For example, solids-reduced DFB product gas may be introduced into syngas compressor 300 for raising the pressure thereof.

Prior to FT synthesis in FT reactor 20, one or more undesirable component(s), such as, but not limited to, sulfur-containing components (e.g. hydrogen sulfide), tar, carbon dioxide, excess hydrogen, and excess carbon monoxide, may be extracted from the compressed DFB product gas in line 305, via conditioning apparatus 10. In embodiments, conditioning apparatus 10 is utilized to reduce the amount of at least one component selected from carbon dioxide, hydrogen (to adjust the molar ratio of hydrogen to carbon monoxide), carbon monoxide (to adjust the molar ratio of hydrogen to carbon monoxide), tar, and hydrogen sulfide, from the compressed DFB product gas introduced thereto via line 305. In embodiments, conditioning apparatus 10 is utilized to reduce the carbon dioxide content of the DFB product gas to a level of less than 20, 15, 10, or 5 volume percent. In embodiments, conditioning apparatus 10 is utilized to reduce the hydrogen sulfide content of the DFB product gas to a level of less than 20, 10, 5, or 1 PPM. In embodiments, conditioning apparatus 10 is utilized to reduce the tar content of the DFB product gas to a level of less than 20, 10, 5, or 1 mg/Nm³. In embodiments, no tar removal other than that provided by catalytic DFB 200 is utilized. In embodiments, no carbon dioxide removal is effected via conditioning apparatus 10.

In embodiments, the herein disclosed method further comprises converting synthesis gas into FT hydrocarbons. An FT feedgas (or ‘FT synthesis gas feed’) is introduced into FT reactor 20 via FT reactor inlet feedgas line 15. As indicated in the Figures, at least a portion of the FT synthesis gas feed is synthesis gas produced/conditioned within catalytic DFB 200. For example, in the embodiment of FIG. 1, the FT feedgas comprises DFB product gas introduced thereto via line 305. In the embodiment of FIG. 1, such DFB product gas is produced via catalytic DFB conditioning of gasification product gas. In the embodiment of FIG. 2, the DFB product gas utilized in the FT syngas feed is produced via catalytic DFB conditioning of a DFB feedgas comprising gasification product gas and optionally also FT tailgas. In the embodiment of FIG. 3, the DFB product gas utilized in the FT syngas feed is produced via catalytic DFB conditioning of a DFB feedgas comprising primarily FT tailgas. In the embodiment of FIG. 3, the FT feed syngas may further comprise additional synthesis gas combined with DFB product gas in line 305 via line 5. The additional synthesis gas in line 5 may have been produced via gasification, reforming, and/or partial oxidation, and may or may not have been conditioned in an other catalytic DFB. In the embodiment of FIG. 4, the DFB product gas is produced via catalytic DFB conditioning of DFB feedgas comprising low and/or medium BTU fuel gas and optionally FT tailgas.

FT reactor 20 is operated as known in the art to produce FT liquid hydrocarbons from the FT syngas feed. An FT overhead is extracted via FT overhead line 26. The FT overhead comprises gaseous light hydrocarbons, unreacted carbon monoxide and hydrogen, carbon dioxide, nitrogen, and other volatilized components. FT product wax (which is molten at the operating temperature of FT reactor 20), is extracted from FT reactor 20 via FT product line 25.

Heat may be recovered from the FT overhead. For example, FT overhead may be extracted from FT reactor 20 via FT overhead line 26 and introduced into FT overhead heat recovery apparatus 30.

Following overhead heat recovery, the reduced temperature overhead may be introduced via line 35 into product separator 40. Product separator 40 is operated to separate a FT tailgas in FT tailgas line 46 from a LFTL product in line 45. The FT tailgas extracted from product separator 40 via FT tailgas line 46 generally comprises unreacted carbon monoxide and hydrogen, carbon dioxide, nitrogen, methane, and other light components. The LFTL product extracted from product separator 40 via LFTL product outlet line 48 comprises light Fischer-Tropsch liquids.

Portions of the raw FT wax in line 25 and/or LFTL in line 48 may be further upgraded as known in the art. For example, raw wax may be introduced into product upgrader 50 via FT product outlet line 25, LFTL may be introduced into product upgrader 50 via LFTL product outlet line 45, or both. Product upgrader 50 may be operated as known in the art to upgrade the materials introduced thereto to provide at least one synthetic fuel. Synthetic fuel product may be extracted from product upgrader 50 via synthetic product outlet line 55. The synthetic fuel may comprise one or more fuel selected from FT naphtha, FT gasoline, FT diesel, and FT jet fuel.

As indicated in the embodiment of FIG. 2, at least a portion of the FT tailgas separated from the LFTL via product separator 40 may be introduced into catalytic DFB 200 via FT tailgas recycle line 46A. The FT tailgas may be recycled to catalytic DFB 200 as a fuel therefor, as a feed thereto, or both. For example, as described in more detail hereinbelow with reference to FIG. 5, FT tailgas may be introduced as a feed into conditioner 210 via FT recycle line 46A′, may be introduced as a fuel into combustor 235 via FT tailgas recycle line 46A″, or both. When the FT tailgas is utilized as both a feed and fuel for catalytic DFB 200, the process may be energy self-sustained (e.g. no supplemental/additional fuel required). As indicated in the embodiment of FIG. 3, and mentioned hereinabove, all or a portion of the FT tailgas may be introduced as the primary DFB feedgas for catalytic DFB 200. In such an embodiment, supplemental fuel may be introduced into catalytic DFB 200 via supplemental fuel line 47. The supplemental fuel may comprise FT tailgas, natural gas, and/or an other fuel. In the embodiment of FIG. 3, an FT tailgas purge may be extracted from DFB 200 via FT tailgas purge line 46B. Such FT tailgas purge and/or utilization of a portion of the FT tailgas or TG purge as fuel for the combustor of the catalytic DFB may be desirable in order to prevent buildup of inerts (e.g. nitrogen). In the embodiment of FIG. 3, compressor 300 serves as a tailgas recycle compressor to compensate for the pressure loss in the FT tailgas recovery system (e.g. product separator 40). In embodiments, substantially all of the FT tailgas is introduced into catalytic DFB 200. In such embodiments, the fuel duty may consist of the supplemental fuel utilized in combustor 235 of catalytic DFB 200, and/or the yield of FT products (e.g. the carbon monoxide conversion to FT products) and/or the conversion of FT tailgas to synthesis gas may be maximized.

As noted in the embodiment of FIG. 4, FT tailgas may be introduced, along with fuel gas in line 270, into catalytic DFB 200 via FT tailgas line 46A. In this embodiment, a portion of the reduced value gas (e.g. low and/or medium BTU fuel gas) may be introduced via line 270A as a fuel for combustor 235 of catalytic DFB 200.

Via embodiments of the herein disclosed system and method incorporating FT tailgas recycle to catalytic DFB, syngas utilization efficiency may be improved. That is, the conversion of synthesis gas into FT products may be increased relative to systems and methods not employing FT tailgas recycle to catalytic DFB. As opposed to conventional processes in which FT tailgas is not passed through a catalytic DFB prior to recycle to FT synthesis apparatus, the FT tailgas recycle in the present case may not have an undesirable (e.g. an undesirably high) molar ratio of hydrogen to carbon monoxide. For example, as discussed further hereinbelow, the DFB product gas may have a molar ratio of hydrogen to carbon monoxide that is suitable for downstream FT synthesis without further adjustment, while that of the FT tailgas may be undesirably high for direct recycle to FT synthesis apparatus 45. In embodiments, the DFB product gas has a molar ratio of hydrogen to carbon monoxide that is suitable for FT processing with an cobalt-based FT catalyst. In embodiments, the DFB product gas has a molar ratio of hydrogen to carbon monoxide that is suitable for FT processing with an iron-based FT catalyst. In embodiments, the DFB product gas has a molar ratio of hydrogen to carbon monoxide that is in the range of from about 0.5:1 to about 5:1, from about 0.5:1 to about 3:1, or from about 0.5:1 to about 2:1.

Operation of Catalytic DFB.

Operation of a catalytic DFB will now be described with reference to FIG. 5. In embodiments, converting non-synthesis gas components of the DFB feedgas into synthesis gas comprises introducing a DFB feedgas into a conditioner/reformer 210 of dual fluid bed conditioning/reformer loop 200. By introducing the conditioner feedgas as a hot gas, reforming may be increased relative to introduction of a cold gas and/or introduction of a hot or cold solid-containing feed (i.e. at least partly solid) directly to reformer/conditioner 210. When utilizing cold, solid feeds, the particles must be broken down, pyrolyzed/volatilized, and then reformed/conditioned. Introducing a hot gas as feed to conditioner 210 may speed up the reforming/conditioning process and increase thermal efficiency relative to other proposed conditioning technologies, in which a gas must first be cooled for processing. Desirably, the feed to the conditioner comprises a substantially homogeneous gas/vapor feed.

Within reformer 210, carbon dioxide, C2+ compounds, and/or methane in the producer gas introduced thereto via line 150 are reformed to produce DFB product synthesis gas. Any reformable low sulfur hydrocarbon bearing vapor or gas may be used as a component of the DFB feedgas to reforming/conditioning reactor 210, including for example unconverted tail gas from a Fischer-Tropsch reactor, as discussed in more detail with reference to the embodiments of FIGS. 2-4. In embodiments, such as that of the embodiment of FIGS. 1 and 2, the DFB feedgas comprises gasification product gas, a method of production of which via a DFB gasifier is described hereinbelow. In the embodiment of FIG. 4, the DFB feedgas comprises fuel gas comprising hydrocarbons and carbon dioxide, as discussed hereinabove.

Reforming is endothermic. To maintain a desired reforming temperature, bed material is circulated to and from combustion reactor 235. A catalytic heat transfer material is circulated throughout dual fluid bed conditioning/reformer loop 200. The material circulated throughout DFB conditioning loop 200 is attrition resistant fluidizable heat transfer material. Desirably, the material is a catalytic material with reforming capability. The catalytic heat transfer material may be supported or unsupported. In embodiments, the catalytic heat transfer material is an engineered material. In embodiments, the catalytic heat transfer material is not engineered. In embodiments, the catalytic heat transfer material comprises a nickel catalyst. In embodiments, the catalytic heat transfer material comprises a supported nickel catalyst. In embodiments, the catalytic heat transfer material comprises a nickel olivine catalyst. In embodiments, the catalytic heat transfer material comprises a supported silica. In embodiments, the catalytic heat transfer material comprises a nickel alumina catalyst. In embodiments, the catalytic heat transfer material is an engineered nickel alumina catalyst. The catalytic heat transfer material may have an particle size distribution in the range of from about 100 microns to about 800 microns, from about 100 to about 600 microns, from about 100 to about 300 microns, about 200 or 100 microns.

In embodiments, the catalytic heat transfer material comprises an engineered alumina support material, which may be from about 10 to about 100 times more attrition resistant than olivine. Such an engineered nickel alumina catalyst may also have a higher heat capacity than olivine. In embodiments, reforming is thus performed with an engineered catalytic support material. In embodiments, the catalytic support material has a high sphericity, wherein the sphericity is defined as the ratio of the surface area of a sphere having the same volume as the particle to the actual surface area of the particle, such that a perfectly spherical particle has a sphericity of 1.0. In embodiments, the sphericity of the engineered support material and/or the catalytic heat transfer material is greater than or equal to about 0.5, 0.6, 0.7, 0.75, 0.85, 0.9, or 0.95. Such an engineered catalytic heat transfer material may be less prone to binding (i.e. flow more readily) throughout DFB conditioning loop 200 (e.g. in cyclone down pipes, cyclone diplegs, and/or in recirculation lines) than non-engineered (i.e. natural) catalytic heat transfer materials (such as olivine-supported materials). Such high sphericity engineered support materials may not only promote reduced particle attrition within a DFB but may also reduce erosion of reaction system components such as refractory, metallic walls, piping, heat exchanger tubing and/or other components. Additionally, the engineered (e.g. engineered alumina) support material may have a higher hardness (e.g. at least about 9.0 on the Mohs scale compared with 6.5 to 7 reported for olivine) and/or higher heat capacity (at least about 0.20 cal/gK at 100° C.) relative to that of natural support materials (e.g. olivine). In embodiments, the catalytic heat transfer material comprises a support having a material density of about 3.6 g/cc. Alpha alumina may be selected over other types of alumina such as gamma alumina because alpha alumina is harder than gamma alumina on the Mohs scale. In embodiments, the BET surface area of the support material is at least about 0.50 m²/g for supported Ni catalyst applications. In embodiments, the nickel content of the catalytic heat transfer material is in the range of from about 1.5 to about 9 weight percent. In applications, the catalytic heat transfer material comprises about 6 weight percent nickel. In applications, the nickel content of the catalytic heat transfer media is substantially less than the typical nickel content of conventional Ni reforming catalysts. In applications, non-supported (homogeneous) Ni based particulate fluidization catalysts based on silica and other substrates are utilized.

If an alumina support material is used as heat transfer media in a primary gasification pyrolysis loop 100 (discussed hereinbelow), a lower BET surface area may be desired, as this may tend to further harden the material, providing greater attrition resistance. The use of an alumina based support material in a gasification pyrolysis loop 100, discussed in detail hereinbelow, may reduce the possibility of agglomeration due to the presence of sodium and/or potassium typically present in biomass feed. The use of silica based support material (sand) or silica containing materials such as natural olivine may tend to form lower melting point eutectics than that of alumina in the presence of sodium and/or potassium, and may thus be less desirable for use in certain applications.

In embodiments, during start-up, thermal activation of an initial batch of catalytic heat transfer material (e.g. a Ni alumina catalyst, for which thermal activation may be performed primarily to decompose residual nitrate content) is effected in situ within secondary combustor 235 without the need for a separate, dedicated activation vessel. Such initial activation may comprise maintaining minimum excess air and/or oxygen levels below 1-2% in spent flue gas line 240 as start-up temperatures exceed 900° F. (482° C.). The reformer/conditioner may be maintained under reducing conditions via a slight hydrogen feed until normal operating feed is introduced after the dual fluid bed reactors have gradually attained normal operational temperatures (e.g. of approximately 1550° F. (843° C.) in the conditioner and approximately 1670° F. (910° C.) to 1700° F. (927° C.) in the combustor). Conventional natural gas and/or propane fuel can be introduced via fuel/tailgas purge line 230 for start-up purposes to gradually elevate system temperatures.

The continuous oxidative regeneration of the catalytic bed material (e.g. engineered nickel alumina catalyst) in combustor 235 within a desired elevated temperature range may also promote resistance to poisoning of the circulating reforming catalyst by residual sulfur compounds which may be present in catalytic DFB feedgas in line 150 or combustor feed in feed line 195. In embodiments, the catalyst utilized as heat transfer material in DFB loop 200 is operable (i.e. retains at least some level of activity) at levels of residual sulfur compounds at least as high as 50, 75, 100, 200, 300, 400, 500, 600, 700, 800, 900 or 1000 ppmv. In embodiments, the catalyst utilized as heat transfer material in DFB loop 200 is operable (i.e. retains at least some level of activity) at levels of residual sulfur compounds at least as high as several hundred ppmv. Generally, as the level of sulfur increases, the activity decreases, as will be discussed further hereinbelow.

In embodiments, reformer/conditioner 210 is operated with H₂S levels of up to at least 50, 75, 80, 90, 100, 150, 200, 300, 400, 500, 600, 700, 800, 900, or up to at least 1000 ppmv, while maintaining at least some catalyst activity as determined by methane conversion. In embodiments, reformer/conditioner 210 is operated with H₂S levels of at least about 150 ppmv, while maintaining substantial catalyst activity. Substantial catalyst activity may comprise methane conversion levels of at least about 50, 75, 90, 95, 96, 97, 98, 99, or substantially 100%. In embodiments, substantial catalyst activity is maintained on a continuous basis for a duration of at least 1, 2, 3, 4, or several hours. In embodiments, catalyst activity lost at high operating levels of sulfur is at least partially regenerated when high sulfur levels in the conditioner or the combustor or throughout DFB conditioning loop 200 are discontinued.

Relatively ‘cold’ bed material is extracted from conditioner/reformer 210 via cold bed material circulation line 225 and introduced into combustion reactor 235. The extracted bed material may comprise uncombusted material, such as coke and unoxidized ash. Within combustion reactor 235, the coke, ash, and/or any other combustible material are combusted with flue gas comprising excess air which is introduced into combustion reactor 230 via flue gas inlet line 195. In embodiments, air/oxidant is introduced into combustor 235 via line 250 which may introduce air directly into combustor 235 or may introduce additional oxidant (e.g. air) into the flue gas 195 exiting combustor 185. Fuel is introduced into combustion reactor 235 via fuel inlet line 230(46K). The fuel may comprise, for example, FT tailgas from a Fischer-Tropsch reactor of downstream processing unit(s) 45. Spent flue gas exits combustion reactor 235 via spent flue gas outlet line 240. Heated bed material is circulated from combustion reactor 235 to conditioner 210 via hot bed material circulation line 215. This circulation of bed material throughout dual fluid bed conditioning loop 200 serves to maintain a desired temperature within conditioner/reformer 210 (i.e. to provide heat thereto via heat transfer with hot circulated materials) and remove unwanted combustible material from the product synthesis gas exiting conditioner 210 via DFB product gas outlet line 220.

In embodiments, the concentration of H₂S in the DFB feedgas in line 150 is at least twice as high as the concentration of SO₂ in flue gas line 195. In embodiments, DFB feedgas introduced into conditioner 210 via DFB feedgas line 150 has a concentration of H₂S of about 100 ppmv, and the concentration of SO₂ in the flue gas introduced into combustor 235 is about 20 ppmv. In embodiments, the total weight of sulfur in the conditioner is approximately the same as the weight of sulfur in the combustor of DFB conditioning loop 200. In embodiments, combustor 235 is operable/operated in the presence of about 0-200 ppmv, about 0-100 ppmv, or about 20-100 ppmv SO₂ in the flue gas feed introduced thereto via line 195, for example, while reformer/conditioner 210 of DFB conditioning loop 200 is able to maintain high activity (e.g. at least about 65, 70, 80, 90, 95, or about 97% catalytic activity).

It should be noted that, in embodiments, not only is reformer/conditioner 210 operable in the presence of H₂S as described above, but this unit may also effectively remove substantially all of the H₂S down to measurable levels of less than about 10, 5, 4, 3, 2 or 1 ppmv in the high quality synthesis gas produced therein (e.g. DFB product gas in line 220), transferring effective sulfur levels to the combustor 235 from which, depending on concentration, it may be released via spent flue gas 240 as SO₂. This may effectively eliminate a need for or reduce size requirements of an H₂S removal system (e.g. a dedicated H₂S removal system) downstream of conditioner 210 and/or upstream of an FT reactor(s) of downstream processing apparatus 45, although, in embodiments, conditioning apparatus 10 is operable to reduce sulfur levels. In embodiments of the herein disclosed method, therefore, a downstream H₂S removal step is absent. Additionally, since SO₂ is less toxic than H₂S and the volume of spent flue gas is generally higher than the volume of high quality synthesis gas, no or reduced size/complexity abatement apparatus or method steps may be needed downstream of combustion/combustor 235 in order to meet local SO₂ emissions regulations, depending on jurisdiction. If further SO₂ abatement is desired, such abatement may, in embodiments, be achieved by dry or wet limestone scrubbing, which may be less costly and/or sensitive to impurities than other forms of conventional H₂S removal. As known in the art, byproduct of dry or wet scrubbing (e.g. calcium sulfate) may be sold for use in the production of various materials, including, but not limited to, sheet rock. Sulfide is more likely to represent the form of sulfur recovery from the gasifier/conditioner; such sulfide may be converted to sulfate in the combustor.

As mentioned hereinabove, the DFB product gas may comprise less than 5, 4, 3, 2, or 1 mg/Nm³ or substantially no tar, while the DFB feedgas may comprise greater than 10, 20, 30, 40, 50, 60, 70, 80, 90, 100, 110, 120, 130, 140 or 150 g/Nm³ tar. In embodiments, substantially all of the tar introduced into conditioner 210 is converted to synthesis gas.

In embodiments, the DFB feed gas to conditioner 210 comprises greater than 5, 10, 15, 20, or 25 volume percent impurities, and the high quality syngas DFB product gas leaving conditioner 210 comprises less than 20, 15, 10 or 8 volume percent impurities (i.e. non-synthesis gas components).

In embodiments, reformer/conditioner 210 is operated at a temperature in the range of from about 1100° F. (593° C.) to about 1600° F. (871° C.), from about 1500° F. (816° C.) to about 1600° F. (871° C.), or from about 1525° F. (829° C.) to about 1575° F. (857° C.), and combustor 235 is operated at a temperature in the range of from about 1600° F. (871° C.) to about 1750° F. (954° C.), from about 1625° F. (885° C.) to about 1725° F. (941° C.), or from about 1650° F. (899° C.) to about 1700° F. (927° C.).

High-quality DFB product synthesis gas is extracted from conditioner/reformer 210 via DFB product gas outlet line 220. In embodiments, the high-quality DFB product syngas comprises low amounts of methane, low amounts of carbon dioxide, and/or low amounts of inerts. In embodiments, the high-quality DFB product synthesis gas comprises less than about 20, less than about 10, or less than about 5 volume percent carbon dioxide. In embodiments, the high-quality synthesis gas comprises less than about 10, 5, or 1 volume percent inerts such as nitrogen. In embodiments, the high-quality synthesis gas comprises less than about 10, 7, or 5 volume percent methane. In embodiments, the high-quality DFB product synthesis gas comprises hydrogen and carbon monoxide in a desired mole ratio. In embodiments, the DFB product gas comprises hydrogen and carbon monoxide in a mole ratio in the range of from about 0.5:1 to about 5:1; alternatively, in the range of from about 0.5:1 to about 1.5:1; alternatively a mole ratio of about 1:1; alternatively a mole ratio of H₂:CO greater than about 1:1. In applications, the DFB product gas is suitable for use in Fischer-Tropsch conversion. In embodiments, the high-quality synthesis gas produced in dual fluid bed conditioning loop 200 requires little or no contaminant removal prior to introduction into a Fischer-Tropsch reactor of downstream processing unit(s) 45, although in embodiments, as described hereinabove, additional conditioning via conditioning apparatus 10 is employed. In applications, the DFB product gas is suitable for direct introduction into a Fischer-Tropsch reactor. In embodiments, H₂S and CO₂ levels are sufficiently low that the high-quality synthesis gas is not introduced into an acid gas removal unit prior to introduction into a Fischer-Tropsch reactor 20 of FT synthesis apparatus 45.

The desired H₂:CO mole ratio and the desired conversion levels of methane, higher hydrocarbons, carbon dioxide, and tars may be achieved primarily by controlling the amount of steam and/or residual water vapor in the feed from which a synthesis gas in the DFB feedgas is produced, (e.g. provided in a biomass feed introduced via carbonaceous feed inlet line 125) introduced into the conditioner with the synthesis gas via line 150 and/or by controlling the operating temperature within conditioner 210. The reforming temperature is ultimately controlled by controlling the rate of circulation of the heat transfer media from combustor 235, while controlling the flow of fuel and/or air or other oxidant to combustor 235 as necessary to maintain a desired combustor temperature.

DFB conditioning loop 200 is operable/utilized for continuous ‘dry reforming’ of methane and/or other hydrocarbons with CO₂ (e.g. a 50/50 molar mix). In embodiments, dry reforming is performed in the presence of tars, with substantially no evidence of catalyst deactivation, and with high (e.g. 90-95+%) molar conversion of the methane, CO₂, and/or tars. In embodiments, a DFB conditioning loop 200 is utilized for efficient dry reforming of propane. In embodiments, the molar ratio of H₂:CO in the DFB product gas may be adjusted to a level of about 1:1 by adjusting the water vapor content of the feed to conditioner 210 introduced via DFB feedgas inlet line 150. Numerous sources and types of hydrocarbons can be efficiently converted to high quality syngas with a desired molar ratio of H₂:CO by varying the steam to carbon molar ratio (i.e. by adjusting steam addition (e.g. introduced into line 150) and/or the degree of drying of a carbonaceous feed from which syngas in the DFB feedgas is produced), without substantial catalyst deactivation and/or coking.

As discussed hereinabove, the amount of steam in conditioner/reformer 210 may be controlled to provide, as DFB product gas, a high quality synthesis gas having a desired composition (e.g. a desired mole ratio of hydrogen to carbon monoxide) and/or a desired degree of tar removal. In applications, the mole ratio of steam (or residual water vapor) to carbon in conditioner 210 is maintained in the range of from about 0.1 to 1. To produce a synthesis gas having a higher mole ratio of hydrogen to carbon monoxide, a mole ratio of steam to carbon may be near the higher end of the range, with more steam being utilized/introduced to conditioner 210. In embodiments, the desired mole ratio of hydrogen to carbon monoxide in the high quality synthesis gas is about 1:1. In such embodiments, the mole ratio of steam to carbon in reformer 210 may be in the range of from about 0.3 to about 0.7; alternatively, in the range of from about 0.4 to about 0.6; alternatively about 0.5. As discussed in more detail hereinbelow, in embodiments, a primary gasification/pyrolysis loop 100 is used to provide low quality producer gas for introduction into conditioner 210 via DFB feedgas inlet line 150. The amount of steam (e.g. low pressure steam having a pressure in the range of from about 25 to about 100 psig (about 1.76 to about 7.03 kg/cm²(g)) introduced into gasification unit 140 via steam inlet line 135 may be adjusted to control the ratio of steam to carbon in conditioner 210. Alternatively or additionally, Fischer-Tropsch tailgas may be utilized in line 135 in addition to some of the steam for fluidization purposes, reducing the amount of steam ending up in conditioner 210. The use of such tail gas or product synthesis gas to minimize steam consumption may be particularly advantageous when the aforementioned ‘indirect’ tubular gasification technologies are used to produce the DFB feedgas for DFB conditioning loop 200 in place of the dual fluidized bed reactors of a gasification DFB loop 100. In such embodiments, a substantial reduction in steam consumption and associated waste water production may occur when such tubular gasification technologies are utilized. Alternatively or additionally, the amount of water in the carbonaceous feed material introduced into a gasification unit 140 via carbonaceous feed inlet line 125 may be adjusted to alter the steam to carbon ratio in conditioner 210.

As mentioned hereinabove, in embodiments, the disclosed method further comprises forming producer gas for introduction into conditioner/reformer 210 via DFB feedgas inlet line 150. Forming of producer gas may be by any means known in the art. However, in an embodiment, the producer gas is formed via the use of a second dual fluid bed loop. In this embodiment, dual fluid bed conditioner/reformer loop 200 is applied as a higher temperature ‘secondary’ DFB reformer loop which receives the corresponding effluent hot gases from a lower temperature ‘primary’ DFB gasification pyrolysis loop 100. As mentioned hereinabove, lower temperature primary DFB gasification loop 100 may gasify any suitable carbonaceous feed, including, but not limited to, biomass (e.g. woody biomass RDF feed), municipal sludge, coal, petroleum coke, and combinations thereof. As indicated in FIG. 5, in such embodiments, conditioner 210 is in series with a gasifier 140, while combustor 235 is in series with combustor 185. Thus, an attrition resistant catalytic (e.g. nickel-based alumina or olivine) DFB conditioning loop may be applied to reforming a poor quality synthesis gas produced by a ‘primary’ DFB gasifier, rather than being applied directly to gasification of carbonaceous feedstock comprising substantial amounts of solids.

In primary gasification loop 100, endothermic primary gasifier 140 pyrolyzes a carbonaceous feed material into synthesis gas in the presence of a suitable fluidizing gas such as steam and/or recycled synthesis gas and/or FT tailgas. In embodiments, use of hydrogen-rich feed promotes lower temperature combustion in fluid bed combustor 185 (e.g. in the range of from about 900° F. (482° C.) to about 1100° F. (593° C.)) than would normally be enabled with hydrocarbon feeds. Thus, in embodiments, hydrogen rich tail gas from an FT synthesis apparatus 45 is introduced via fuel/tailgas purge line 180 (46B) to facilitate lower temperature operation of combustor 185 of a lower temperature gasification pyrolysis loop 100.

The carbonaceous feed material may be primarily solid, primarily liquid, primarily gaseous, or may contain any combination of solid, liquid and gaseous carbonaceous materials. In embodiments, the carbonaceous feed is in the form of a slurry. In applications, the carbonaceous feed material introduced into gasifier 140 via carbonaceous feed inlet line 125 comprises or is derived from RDF, municipal sludge, biomass, coal, petroleum coke or a combination thereof. Suitable processed municipal sludge comprises, for example, E-FUEL™, available from Enertech, Atlanta, Ga. In applications, the carbonaceous feed comprises primarily RDF. In embodiments, bulk feed material is introduced into a feedstock (e.g. an at least partially solid feedstock) collection bin of carbonaceous handling apparatus 90. Feed may be introduced from the feedstock collection bin into a screw feeder. The carbonaceous feed material is introduced into gasifier 140 of gasification DFB loop 100 via carbonaceous feed material inlet line 125. As mentioned hereinabove, liquid or high sulfur vapor hydrocarbons may be introduced into gasifier 140 via line 130. In this manner, high sulfur-containing materials may be converted to synthesis gas, and the sulfur effectively removed from the DFB product synthesis gas.

Any unconverted char produced in gasifier 140 is oxidized with oxidant (e.g. air) in exothermic primary combustor 185. As shown in FIG. 5, routing all of the system combustion air requirements through primary combustor 185 may be used to promote complete combustion in primary combustion reactor 185, even though the combustor is desirably operated at lower temperatures than combustor 235. In embodiments, however, a portion of oxidant (e.g. air) from line 175 is routed directly to combustor 235, for example via line 250. Gasification loop 100 utilizes any suitable circulating heat transfer medium to transfer heat from primary combustor 185 to gasifier 140. As mentioned hereinabove, the heat transfer medium may be silica, olivine, alumina, or a combination thereof. The introduction of excess air into primary combustor 185 via line 175, permits operation of combustor 185 at lower temperature while achieving high char combustion. Such lower operating combustion temperature may help suppress production of undesirables, such as, but not limited to, thermal NOx and/or dioxin production and reduction thereof in the flue gas which ultimately exits catalytic DFB 200 via spent flue gas outlet line 240. The lower temperature operation of gasification DFB loop 100 may enable enhanced contaminant removal, as mentioned hereinabove. The poorer ‘low’ quality synthesis gas produced in gasification unit 140 is reformed in catalytic DFB loop 200, providing ‘high quality’ synthesis gas of a desired composition (e.g. having a desired H₂:CO mole ratio and/or a desired purity). In applications, operation of combustor 235 at a higher temperature than combustor 185 permits combustion of any residual hydrocarbons carried over from gasification DFB loop 100, including highly toxic hydrocarbons, such as dioxins and PCBs which may be present.

In embodiments, combustor 235 is operated with less than or equal to about 5, 4, 3, 2, 1, or 0.5 volume percent oxygen, and/or less than or equal to about 2, 1, or 0.5 volume percent carbon monoxide in spent flue gas stream 240. In embodiments, combustor 235 is operated with less than about 1 volume percent oxygen and less than about 0.5 volume percent carbon monoxide in spent flue gas stream 240. In embodiments, combustor 235 is operated with approximately (e.g. slightly above) stoichiometric air. In embodiments, combustor 235 is operated with from about 1 to about 1.1 stoichiometric air. In embodiments, combustor 235 is operated with less than or equal to about 1.1, 1.05, or 1 times stoichiometric air. In embodiments, low excess oxygen levels are utilized to prevent/minimize carryover of oxygen in catalytic heat transfer material (e.g. with Ni catalyst) exiting combustor 235 via line 215 to the reformer/conditioner of DFB conditioning loop 200. Excess oxygen may not be desirable because it leads to increased levels of CO₂ in the high quality syngas in line 220 (which must be removed prior to certain applications requiring chemical grade synthesis gas) and also reduces synthesis gas yield (defined as moles of CO plus H₂). Reducing circulation rates between the reactors of DFB conditioning loop 200 may also be utilized to prevent undesirable oxygen carryover. Quite unexpectedly, a DFB system originally designed for oxygen carryover has been successfully applied to an application in which oxygen carryover is undesirable.

In embodiments, another advantage of operating with the substantially zero excess air consumption enabled by secondary combustor 235 in conditioning DFB loop 200 is more complete utilization of the unconverted excess air in the flue gas exiting primary combustor 195 of the primary gasification pyrolysis loop 100, as typified by more conventional indirect gasifier concepts, such as those of SilvaGas and Clearfuels. Not only does this potentially minimize the size and/or power consumption of an air compressor providing oxidant to combustor 185 and associated processing equipment, such operation may also reduce pollutant production (e.g. NOx and/or dioxin production) within spent flue gas leaving the system via line 240 compared with prior art systems. The high efficiency of flue gas oxygen utilization in secondary combustor 235 may also facilitate efficient use of other low grade flue gas sources as a supplemental feed to primary combustor 185 and/or secondary combustor 235. Such supplemental feed may comprise exhaust gas from a gas turbine, for example, which may comprise substantial amounts of oxygen and may be introduced from a gas turbine exhaust line fluidly connected via line 265 and/or line 260 into combustor 235 and/or primary combustor 185. Such exhaust gas may be introduced ‘hot’, thus reducing energy requirements.

If the feed to primary gasifier 140 contains significant levels of sulfur and/or halogen (e.g. chlorine), a suitable contaminant-removal compound, such as limestone, dolomite or calcined lime (CaO), and/or sodium carbonate, may be added to gasification loop 100 to prevent excessive levels of contaminant compounds (e.g. sulfur and/or halogen) from contaminating the effluent gases in conditioner inlet line 150 entering catalytic DFB conditioning loop 200. The resulting byproduct (e.g. calcium sulfate and/or calcium halide) along with any ash introduced with the primary loop gasification feed via carbonaceous feed inlet line 125 may be purged from the heat transfer medium leaving primary combustor 185 in ‘hot’ bed material circulation line 155, for example, via purge line 160. Capturing chlorine, via for example use of a nickel alumina catalyst or other suitable material, in primary DFB gasification loop 100 may reduce the likelihood of dioxin production.

Some synthetic or engineered catalyst support materials, such as CoorsTek alumina for example, may be recyclable following appropriate processing. Such processing may involve, for example, the addition of appropriate binder material to reagglomerate the fines and spray drying to reconstitute the originally desired particle size distribution. In embodiments, the desired particle size distribution is in the range of from about 100 to about 800 microns, from about 100 to about 600 microns, from about 100 to about 400 microns or from about 100 to about 300 microns. The reconstituted support material could subsequently undergo the usual processing for Ni catalyst addition to render it reusable and recyclable as catalyst to the Ni DFB system. While minimizing the process make-up requirement for fresh catalyst material, which may be costly, such catalyst reconstitution may also help minimize the potential disposal burden of spent nickel-contaminated catalyst. Such recycling could represent another advantage of utilizing/selecting an engineered catalyst support material rather than a conventional material such as natural olivine, which may not be recyclable in this manner.

Desirably, the circulating heat transfer media in both continuous regenerative DFB loops 100 and 200 (e.g. catalytic heat transfer medium in catalytic DFB 200 and silica, olivine and/or alumina heat transfer medium in gasification loop 100) are operated independently of one another, whereby cross contamination of any catalysts, heat transfer media, adsorbents, and/or other additives is minimized. Each continuous regenerative loop 100 and 200 may therefore be optimized to maximize individual performance levels and individual feedstock flexibility of the respective loop, while achieving the important thermal efficiency advantage of integrated hot gas processing, an industry first.

By utilizing primary gasification and secondary conditioning, gasification may be operated at lower temperatures than conditioning (e.g. reforming). In this manner, greater amounts of undesirables, e.g. sulfur-containing components, may be absorbed and removed via the lower temperature gasification loop. Such absorption of undesirables tends to work better at reduced temperatures. The gasification stage may thus perform more efficiently and reliably at lower operating temperatures with regard to sulfur capture and other parameters as described in this disclosure, with concomitant increased flexibility/range of suitable carbonaceous feedstocks.

In embodiments, substantially all (up to 99.9% or more) of any residual low levels of carbonyl sulfide and/or other acid gases such as H₂S remaining in the DFB product gas exiting catalytic DFB 200 in DFB product gas outlet line 220 may be removed downstream of catalytic DFB 200, for example, via a conventional caustic scrubber of conditioning apparatus 10, optionally following heat recovery and gas cooling in heat recovery apparatus 500.

While an embodiment of the invention has been described in which the catalytic DFB 200 of this disclosure is applied as a secondary loop to a primary DFB gasifier loop 100, as noted hereinabove, the method of producing high-quality DFB product synthesis gas via catalytic DFB 200 can be integrated with similarly high thermal efficiency with other types of ‘indirect’ gasification technologies in which air is indirectly used as a gasification (combustion) agent without diluting the synthesis gas produced with the nitrogen content of the air and resulting flue gas. These other types of indirect gasification technologies include biomass (e.g. low sulfur biomass) to Fischer-Tropsch liquids (BTL) applications. Substantial BTL yield improvement may result if the conditioning method disclosed herein is similarly applied to the synthesis gas and flue gas effluents from these technologies. Gasification feeds comprising higher levels of sulfur may be utilizable if a desulfurizing agent (e.g. a lime-based desulfurizing agent) is added to the selected gasifier (e.g. a fluid bed gasifier).

In embodiments, ‘direct’ gasification technologies that provide synthesis gas having suitably low sulfur content are utilized to provide at least a portion of the DFB feedgas introduced into catalytic DFB 200 via DFB feedgas inlet line 150. Direct fluid bed gasification technologies may also be capable of gasifying higher sulfur feedstocks if it is also feasible to add a desulfurizing agent (e.g. a lime-based desulfurizing agent) to the gasifier.

By integrating the disclosed catalytic dual fluid bed conditioning method with existing biomass to liquids (BTL) and/or coal to liquids (CTL) applications, large yield and cost improvements may be realized. The system and method disclosed herein allow hot gas processing, eliminating the need for costly low temperature or cryogenic processes and apparatus.

Utilization of a lower temperature gasification loop 100 may pyrolyze, de-ash, desulfurize and/or dehalogenize low quality carbonaceous feedstocks, while a higher temperature catalytic DFB loop 200 efficiently reforms the resulting methane, other light hydrocarbons, and any CO₂ into high quality DFB product synthesis gas. The conditioning (e.g. reforming) reactions occur more efficiently in the absence of unconverted solid feedstock or associated ash residues, which could hinder the efficient gas phase mass transfer and kinetics of the reforming reactions. Both DFB loops may be continuously and independently regenerated via segregated oxidant-blown (e.g. air-blown) combustion of the respective circulating heat transfer and/or catalytic media of that loop. The serial hot gas processing configuration of the corresponding primary and secondary reactors maximizes thermal efficiencies therein, while substantially reducing or even eliminating the need for intervening heat transfer equipment. Segregating and optimizing the individual dual fluid bed pyrolysis and reforming operations in the unique serial configurations described herein may result in more efficient utilization of steam, catalyst, feedstocks, and fuel for high quality synthesis gas production than described in the art.

### EXAMPLES

### Example 1

### NiDFB Testing

Tests of the operation of a nickel DFB were performed to verify the effectiveness thereof to dry reform methane and carbon dioxide to produce synthesis gas. Dry reforming of carbon dioxide and methane was effected even in the presence of trace contaminants, such as sulfur. In Table 2, AR refers to the combustor/regenerator 235 of the NiDFB 200, while FR refers to reformer/conditioner 210 thereof. The preferred gas comprised tailgas and synthesis gas, the surrogate gas comprised tailgas, carbon dioxide and water, and the CH4 Rich 5 gas comprised methane and carbon dioxide.

TABLE 2
Results of Dry Reforming of Example 1.

| | | FR | | | | Seal | | |
|---|---|---|---|---|---|---|---|---|
| | Feed Gas | Feed | FR Feed Contambiants | | | Loop | | |
| | Used (Notes | Flow | Tars | | H₂S | N₂ | FR | AR |
| Date | 1, 3) | Nm3/hr | mg/Nm3 | ppmv | ppmv | Nm3/hr | T (° C.) | T (° C.) |
| Experiment 1 | | | | | | | | |
| 29-Jul, 8P | Surrogate | 25.00 | | | | 5 | 855 | 890 |
| 29-Jul, 9P | Preferred | 25.00 | | | | 5 | 855 | 890 |
| 30-Jul, 4A | CH4 Rich 5 | 25.00 | | | | 7 | 850 | 890 |
| 30-Jul, 5A | CH4 Rich 5 | 27.00 | | | | 7 | 845 | 885 |
| Experiment 2 | | | | | | | | |
| 11-Aug, 8P | Surrogate | 25.00 | | | | 5 | 850 | 900 |
| 11-Aug, 4P | Surrogate | 23.00 | | | | 5 | 900 | 935 |
| 11-Aug, 5P | Surrogate | 25.22 | 35,890 | 8,700 | | 5 | 890 | 980 |
| 11-Aug, 9P | Surrogate | 25.22 | 35,890 | 8,700 | | 5 | 855 | 890 |
| 12-Aug, 2A | CH4 Rich 5 | 15.43 | 116,618 | 28,999 | | 5 | 855 | 895 |
| 12-Aug, 4P | Surrogate | 25.21 | 35,894 | 8,574 | | 5 | 850 | 895 |
| 12-Aug, 8P | CH4 Rich 5 | 10.00 | | | | 5 | 795 | 845 |
| 12-Aug, 9P | CH4 Rich 5 | 10.07 | 29,784 | 7,250 | | 5 | 790 | 840 |

Experiment 3
(See Note 2)

| | | | | | | | | |
|---|---|---|---|---|---|---|---|---|
| 19-Aug, 1P | Surrogate | 25.00 | | | | 7 | 850 | 880 |
| 19-Aug, 9P | Surrogate | 15.00 | | | 82 | 5 | 848 | 893 |
| 20-Aug, 8A | Surrogate | 15.00 | | | 161 | 5 | 875 | 928 |
| 20-Aug, 7P | Surrogate | 15.12 | 32,007 | 8,092 | 160 | 5 | 855 | 912 |

| | | AR Flue Gas | | FR Product Gas Comp, | | | | | | |
|---|---|---|---|---|---|---|---|---|---|---|
| | Feed Gas | Est | | Mol % (Dry & inert Free) | | | | | | Est |
| | Used (Notes | CO | O₂ | H₂ | CO | CO₂ | CH₃ | Total | H₂/ | CH₄ |
| Date | 1, 3) | Vol % | | Vol % | | | | | CO | Conv |
| Experiment 1 | | | | | | | | | | |
| 29-Jul, 8P | Surrogate | 1 | 0 | 44.42 | 43.55 | 11.53 | 0.00 | 100.00 | 1.02 | Not |
| 29-Jul, 9P | Preferred | 1 | 0 | 45.45 | 43.00 | 11.55 | 0.00 | 100.00 | 1.06 | Calc |
| 30-Jul, 4A | CH4 Rich 5 | 1 | 0 | 48.87 | 50.32 | 5.16 | 0.65 | 100.00 | 0.87 | |
| 30-Jul, 5A | CH4 Rich 5 | 1 | 0 | 46.54 | 46.54 | 6.29 | 0.63 | 100.00 | 1.00 | |
| Experiment 2 | | | | | | | | | | |
| 11-Aug, 8P | Surrogate | 1 | 0 | 46.43 | 42.88 | 10.71 | 0.00 | 100.00 | 1.08 | Not |
| 11-Aug, 4P | Surrogate | 1 | 0 | 42.55 | 46.10 | 11.35 | 0.00 | 100.00 | 0.92 | Calc |
| 11-Aug, 5P | Surrogate | 1.4 | 0 | 41.67 | 48.61 | 9.72 | 0.00 | 100.00 | 0.86 | |
| 11-Aug, 9P | Surrogate | 0.6 | 0 | 41.38 | 48.28 | 9.65 | 0.89 | 100.00 | 0.85 | |
| 12-Aug, 2A | CH4 Rich 5 | 0.3 | 0 | 43.75 | 51.25 | 3.13 | 1.88 | 100.00 | 0.85 | |
| 12-Aug, 4P | Surrogate | 0.5 | 0 | 41.67 | 45.83 | 11.11 | 1.89 | 100.00 | 0.91 | |
| 12-Aug, 8P | CH4 Rich 5 | 0.6 | 0 | 46.45 | 46.45 | 3.23 | 8.87 | 100.00 | 1.00 | |
| 12-Aug, 9P | CH4 Rich 5 | 0.5 | 0 | 41.38 | 48.28 | 5.52 | 4.83 | 100.00 | 0.85 | |
| Experiment 3 | | | | | | | | | | |
| (See Note 2) | | | | | | | | | | |
| 19-Aug, 1P | Surrogate | 0.5 | 0 | 42.99 | 48.44 | 19.57 | 0.00 | 100.00 | 0.99 | 100.00 |
| 19-Aug, 9P | Surrogate | 0.35 | 0.15 | 41.13 | 42.81 | 18.89 | 2.17 | 100.00 | 0.96 | 91.00 |
| 20-Aug, 8A | Surrogate | 0.2 | 0.05 | 40.94 | 42.53 | 19.57 | 0.96 | 100.00 | 0.95 | 95.30 |
| 20-Aug, 7P | Surrogate | 0.3 | 0 | 41.67 | 45.66 | 10.76 | 1.91 | 100.00 | 0.91 | 93.10 |

Note 1—See Table 2a, Feed gas compositions
Note 2—Calibration error reported for CO2 levels, TUV to correct
Note 3—2 Nm³/h of steam added for the July-30. SP test

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g. from about 1 to about 10 includes, 2, 3, 4, etc.; greater than 0.10 includes 0.11, 0.12, 0.13, and so forth). Use of the term ‘optionally’ with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope protection is not limited by the description set out above but is only limited by the claims which follow, that scope including all equivalents of the subject matter of the claims. Each and every original claim is incorporated into the specification as an embodiment of the present invention. Thus, the claims are a further description and are an addition to the preferred embodiments of the present invention. The disclosures of all patents, patent applications and publications cited herein are hereby incorporated herein by reference, to the extent that they provide procedural or other details consistent with and supplementary to those set forth herein.

What is claimed is:

1. A system for the production of synthetic fuel, the system comprising:
a catalytic dual fluidized bed (DFB) configured to produce, from a DFB feedgas, a DFB product comprising synthesis gas; and
a Fischer-Tropsch (FT) synthesis apparatus fluidly connected with the catalytic DFB, wherein the FT synthesis apparatus comprises:
an FT synthesis reactor configured to produce, from an FT feedgas, an FT overhead and a liquid FT product comprising FT wax, wherein the FT feedgas comprises at least a portion of the DFB product; and
a product separator downstream of and fluidly connected with the FT synthesis reactor, wherein the product separator is configured to separate, from the FT overhead, an FT tailgas and an LFTL product comprising LFTL.

2. The system of claim 1 further comprising a fluid connection between the product separator and the catalytic DFB, whereby at least a portion of the FT tailgas can be introduced into the catalytic DFB.

3. The system of claim 2 further comprising one or more apparatus selected from the group consisting of:
gasification apparatus configured to produce synthesis gas from a gasifier feed;
compressors upstream of the FT synthesis reactor and configured to compress at least a portion of the FT feedgas;
syngas conditioning apparatus selected from the group consisting of tar removal apparatus, CO₂ removal apparatus, sulfur removal apparatus, and combinations thereof, wherein the syngas conditioning apparatus is located upstream of and is fluidly connected with the FT synthesis reactor;
heat recovery apparatus downstream of and fluidly connected with the catalytic DFB and configured to recover heat from the DFB product gas;
heat recovery apparatus downstream of and fluidly connected with the FT synthesis reactor and configured to recover heat from the FT overhead;
solid/gas separators upstream of the catalytic DFB and configured to remove solids from at least a portion of the DFB feedgas;
solid/gas separators downstream of the catalytic DFB and configured to remove solids from at least a portion of the DFB product gas; and
product upgrading apparatus downstream of and fluidly connected with the product separator, wherein the product upgrading apparatus is configured to upgrade at least a portion of the LFTL product, at least a portion of the liquid FT product, or at least a portion of both the LFTL product and the liquid FT product, thus providing one or more synthetic fuels.

4. The system of claim 3 wherein the syngas conditioning apparatus comprises no tar removal apparatus.

5. The system of claim 4 comprising at least one of each of the apparatus listed therein, and wherein the gasification apparatus comprises an indirect biomass gasifier and is fluidly connected with the catalytic DFB.

6. The system of claim 2 configured for introduction of the at least a portion of the FT tailgas into the catalytic DFB as a fuel, as a feedgas, or both.

7. The system of claim 1 wherein the catalytic DFB comprises
a fluid bed conditioner operable to produce the DFB product gas from the DFB feedgas, wherein the fluid bed conditioner comprises an outlet for a first catalytic heat transfer stream comprising a catalytic heat transfer material and having a first temperature, and an inlet for a second catalytic heat transfer stream comprising catalytic heat transfer material and having a second temperature that is greater than the first temperature;
a fluid bed combustor operable to combust fuel and oxidant introduced thereto, wherein the fluid bed combustor comprises an inlet fluidly connected with the outlet for a first catalytic heat transfer stream of the conditioner, and an outlet fluidly connected with the inlet for a second catalytic heat transfer stream of the fluid bed conditioner; and
a catalytic heat transfer material

8. The system of claim 7 wherein the catalytic heat transfer material comprises a supported or unsupported metal catalyst.

9. The system of claim 8 wherein the catalytic heat transfer material comprises a supported or unsupported nickel catalyst.

10. The system of claim 8 wherein the catalytic heat transfer material comprises a supported catalyst, and wherein the support is selected from the group consisting of alumina, olivine, silica, and combinations thereof.

11. The system of claim 7 wherein the DFB feedgas comprises a low quality synthesis gas, wherein the low quality synthesis gas comprises a greater percentage of non-syngas components than the DFB product gas, and wherein the system further comprises a gasifier operable to produce the low quality synthesis gas, wherein the gasifier is located upstream of the fluid bed conditioner and fluidly connected therewith, whereby at least a portion of the low quality synthesis gas may be introduced into the fluid bed conditioner as DFB feedgas.

12. The system of claim 11 wherein the gasifier is one fluid bed of a dual fluidized bed gasification apparatus.

13. The system of claim 12 wherein the dual fluidized bed gasification apparatus comprises:
a fluid bed gasifier operable to produce low quality synthesis gas from carbonaceous material and optionally steam, and comprising an outlet for a first heat transfer stream comprising a heat transfer material and unconverted carbonaceous material and having a third temperature, and an inlet for a second heat transfer stream comprising heat transfer material and having a fourth temperature greater than the third temperature;
a second fluid bed combustor operable to combust oxidant and fuel and produce a flue gas, wherein the second fluid bed combustor comprises a second fluid bed combustor inlet fluidly connected with the outlet for a first heat transfer material stream of the fluid bed gasifier, and a second fluid bed combustor outlet fluidly connected with the inlet for a second heat transfer stream of the fluid bed gasifier; and
a heat transfer material.

14. The system of claim 13 wherein the carbonaceous material is selected or derived from a material selected from the group consisting of biomass, municipal sludge, RDF, coal, petroleum coke, natural gas, E-FUEL, and combinations thereof.

15. The system of claim 7 further comprising a fluid connection between the fluid bed conditioner and the product separator, whereby at least a portion of the FT tailgas can be introduced into the fluid bed conditioner as at least one carbon-containing component of the DFB feedgas.

16. The system of claim 15 configured such that the DFB feedgas comprises substantially no carbon-containing gas other than the FT tailgas.

17. The system of claim 16 wherein the FT tailgas comprises carbon dioxide and at least one component selected from methane, ethane, propane, and higher hydrocarbons, and wherein the catalytic DFB is operable to continuously dry reform the DFB feedgas to produce the DFB product comprising synthesis gas.

18. The system of claim 15 configured for the introduction of additional synthesis gas, not produced in the catalytic DFB, into the FT synthesis reactor, whereby the additional synthesis gas and the at least a portion of the DFB product gas can be introduced into the FT synthesis reactor as FT feedgas.

19. The system of claim 18 wherein the additional synthesis gas is produced via gasification, reforming, partial oxidation, or a combination thereof.

20. The system of claim 18 further comprising one or more apparatus selected from the group consisting of:
compressors upstream of the FT synthesis reactor and configured to compress at least a portion of the FT feedgas;
heat recovery apparatus downstream of and fluidly connected with the FT synthesis reactor and configured to recover heat from the FT overhead; and
product upgrading apparatus downstream of and fluidly connected with the product separator, wherein the product upgrading apparatus is configured to upgrade at least a portion of the LFTL product, at least a portion of the liquid FT product, or at least a portion of both the LFTL product and the liquid FT product, thus providing one or more synthetic fuels.

21. The system of claim 20 comprising at least one of each of the apparatus listed therein.

22. The system of claim 1 further comprising one or more apparatus selected from the group consisting of:
gasification apparatus configured to produce synthesis gas from a gasifier feed;
compressors upstream of the FT synthesis reactor and configured to compress at least a portion of the FT feedgas;
syngas conditioning apparatus selected from the group consisting of tar removal apparatus, CO₂ removal apparatus, sulfur removal apparatus, and combinations thereof, wherein the syngas conditioning apparatus is located upstream of and is fluidly connected with the FT synthesis reactor;
heat recovery apparatus downstream of and fluidly connected with the catalytic DFB and configured to recover heat from the DFB product gas;
heat recovery apparatus downstream of and fluidly connected with the FT synthesis reactor and configured to recover heat from the FT overhead;
solid/gas separators upstream of the catalytic DFB and configured to remove solids from at least a portion of the DFB feedgas;
solid/gas separators downstream of the catalytic DFB and configured to remove solids from at least a portion of the DFB product gas; and
product upgrading apparatus downstream of and fluidly connected with the product separator, wherein the product upgrading apparatus is configured to upgrade at least a portion of the LFTL product, at least a portion of the liquid FT product, or at least a portion of both the LFTL product and the liquid FT product, thus providing one or more synthetic fuels.

23. The system of claim 22 wherein the syngas conditioning apparatus comprises no tar removal apparatus.

24. The system of claim 23 comprising at least one of each of the apparatus listed therein, and wherein the gasification apparatus comprises an indirect biomass gasifier and is fluidly connected with the catalytic DFB.

25. The system of claim 1 configured for the introduction into the catalytic DFB of a DFB feedgas comprising one or more gas selected from the group consisting of low BTU fuel gases and medium BTU fuel gases, and wherein the catalytic DFB is operable to continuously dry reform the DFB feedgas to produce the DFB product comprising synthesis gas.

26. The system of claim 25 wherein the DFB feedgas consists essentially of no other carbon-containing gas other than one or more gas selected from the group consisting of low BTU fuel gases and medium BTU fuel gases, and FT tailgas.

27. A method of producing synthetic fuel, the method comprising:
producing a dual fluidized bed (DFB) product from a DFB feedgas, via a catalytic DFB, wherein the DFB product comprises synthesis gas;
introducing an FT feedgas comprising at least a portion of the DFB product into an FT synthesis reactor;
extracting a gaseous FT overhead and a liquid FT product comprising FT wax from the FT synthesis reactor;
separating, from the FT overhead, an FT tailgas and an LFTL product comprising LFTL; and
upgrading at least a portion of the LFTL product, at least a portion of the liquid FT product, or at least a portion of both the LFTL product and the liquid FT product, thus providing one or more synthetic fuels.

28. The method of claim 27 further comprising introducing at least a portion of the FT tailgas into the catalytic DFB.

29. The method of claim 28 wherein the at least a portion of the FT tailgas is introduced into the catalytic DFB as a fuel, as at least a component of the DFB feedgas, or both.

30. The method of claim 27 wherein producing a DFB product from a DFB feedgas further comprises introducing the DFB feedgas into a fluid bed conditioner, wherein the fluid bed conditioner is configured to convert at least a portion of said DFB feedgas into synthesis gas;
extracting a first catalytic heat transfer stream comprising a catalytic heat transfer material and having a first temperature from the fluid bed conditioner and introducing at least a portion of the first catalytic heat transfer stream and a flue gas into a fluid bed combustor, wherein the fluid bed combustor is configured to regenerate the catalyst;
extracting a second catalytic heat transfer stream comprising catalytic heat transfer material and having a second temperature from the fluid bed combustor and introducing at least a portion of the second catalytic heat transfer stream into the fluid bed conditioner; and
extracting the DFB product from the fluid bed conditioner.

31. The method of claim 30 wherein the catalytic heat transfer material comprises a supported or unsupported metal catalyst.

32. The method of claim 31 wherein the catalytic heat transfer material comprises a supported or unsupported nickel catalyst.

33. The method of claim 31 wherein the catalytic heat transfer material comprises a supported catalyst, and wherein the support is selected from the group consisting of alumina, olivine, silica, and combinations thereof.

34. The method of claim 30 further comprising introducing at least a portion of the FT tailgas into the fluid bed conditioner as at least a component of the DFB feedgas.

35. The method of claim 34 wherein the FT feedgas further comprises additional synthesis gas not produced in the catalytic DFB.

36. The system of claim 35 further comprising producing the additional synthesis gas via gasification, reforming, partial oxidation, or a combination thereof.

37. The method of claim 34 wherein the DFB feedgas comprises substantially no carbon-containing gas other than the FT tailgas.

38. The method of claim 37 wherein the FT tailgas comprises carbon dioxide and at least one component selected from the group consisting of methane, ethane, propane, and higher hydrocarbons, and wherein the catalytic DFB is operable to continuously dry reform the DFB feedgas to produce the DFB product comprising synthesis gas.

39. The method of claim 30 further comprising producing low quality synthesis gas by gasifying a carbonaceous material, and wherein the DFB feedgas comprises at least a portion of the low quality synthesis gas.

40. The method of claim 39 wherein the carbonaceous material is derived from or selected from the group consisting of biomass, municipal sludge, RDF, coal, petroleum coke, natural gas, E-FUEL and combinations thereof.

41. The method of claim 39 wherein gasifying a carbonaceous material comprises:
introducing the carbonaceous material into a fluid bed gasifier of a dual fluidized bed gasification apparatus, wherein the carbonaceous material is gasified under gasification conditions;
extracting a first heat transfer stream comprising heat transfer media and any unconverted carbonaceous material from the fluid bed gasifier and introducing at least a portion of the first heat transfer stream into a second fluid bed combustor, wherein the first heat transfer stream has a third temperature;
introducing oxidant and fuel into the second fluid bed combustor whereby unconverted carbonaceous material in the first heat transfer stream is combusted and the temperature of the heat transfer media is raised;
extracting a second heat transfer stream comprising heat transfer media and having a fourth temperature that is greater than the third temperature from the second fluid bed combustor and introducing at least a portion of the second heat transfer stream into the fluid bed gasifier; and
extracting low-quality synthesis gas from the fluid bed gasifier.

42. The method of claim 30 further comprising operating the fluid bed combustor at from about 1 to 1.1 times stoichiometric air.

43. The method of claim 27 further comprising removing at least one component selected from the group consisting of tar, carbon dioxide, and sulfur from the at least a portion of the DFB product prior to introduction thereof into the FT synthesis reactor.

44. The method of claim 43 comprising no additional tar removal from the at least a portion of the DFB product prior to introduction thereof into the FT synthesis reactor.

45. The method of claim 27 wherein the DFB feedgas comprises one or more gas selected from the group consisting of low BTU fuel gases and medium BTU fuel gases, and wherein the catalytic DFB is operable to continuously dry reform the DFB feedgas to produce the DFB product comprising synthesis gas.

46. The method of claim 45 wherein the DFB feedgas comprises no carbon-containing gas other than one or more carbon-containing gas selected from the group consisting of low BTU fuel gases, medium BTU fuel gases, FT tailgas, and combinations thereof.
